SEQUENCE LISTING (10 sequences)

SEQ ID NO: 1 - protein, 9 aa, Artificial
  Cytomegalovirus pp89 epitope
YPHFMPTML

SEQ ID NO: 2 - protein, 9 aa, Artificial
  LCMV nucleoprotein epitope
RPQASGVYM

SEQ ID NO: 3 - protein, 9 aa, Artificial
  Influenza virus nucleoprotein epitope
TYQRTRALV

SEQ ID NO: 4 - protein, 9 aa, Plasmodium berghei circumsporozoite protein epitope
SYIPSAEKI

SEQ ID NO: 5 - protein, 9 aa, Artificial
  HIV gag epitope
AMQMLKETI

SEQ ID NO: 6 - protein, 8 aa, Artificial
  Human Ovalbumin Epitope
SIINFEKL

SEQ ID NO: 7 - DNA, 28 bp, Artificial
  mpt polytope F primer
gcgacgcgtctagagccagcaacgagaa

SEQ ID NO: 8 - DNA, 27 bp, Artificial
  mpt polytope R primer
gtaacgcgtctaagtcctcggggccgg

SEQ ID NO: 9 - DNA, 25 bp, Artificial
  HIVgag primer
accatgggcgcgagcatcggtatta

SEQ ID NO: 10 - DNA, 27 bp, Artificial
  HIV gag primer
ctaaagcgcgccttgtgacgaggggtc

(19) United States
(12) Patent Application Publication
(10) Pub. No.: US 2006/0204523 A1
(43) Pub. Date: Sep. 14, 2006

(54) Flavivirus vaccine delivery system

(76) Inventors: Alexander A Khromykh, The Gap (AU); Andreas Suhrbier, Bunya (AU)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 10/496,421

(22) PCT Filed: Nov. 26, 2002
(86) PCT No.: PCT/AU02/01598

§ 371 Date: Feb. 17, 2005

(30) Foreign Application Priority Data
Nov. 26, 2001 (AU) PR 9093
Jul. 29, 2002 (AU) 2002950974

(51) Int. Cl. A61K 39/12; A61K 39/193; C12N 15/86

(52) U.S. Cl. 424/218.1; 435/456; 435/325

(57) ABSTRACT

A flaviviral expression vector, construct and system are provided that each comprise a flaviviral replicon that faciliates intracellular self-replication leading to high-level expression of RNA and protein(s) encoded by a heterologous nucleic acid. The flaviviral expression vector, construct and system preferably use a Kunjin virus replicon and are of particular use as a vaccine delivery system that facilitates expression of immunogenic proteins and peptides that induce protective T cell immunity to viral infections and cancer. Vaccines may be administered in the form of DNA, RNA or virus like particles.

### FIELD OF THE INVENTION

THIS INVENTION relates to an improved flaviviral replicon, expression vector, construct and system that comprises said flaviviral replicon. More particularly, this invention relates to a flaviviral expression system useful as a vaccine delivery system encoding heterologous, immunogenic proteins and peptides that induce protective T cell immunity to viral infections and cancer. Vaccines may be administered in the form of DNA, RNA or virus like particles whereby intracellular self-replication leads to high-level expression of RNA and encoded protein(s).

### BACKGROUND OF THE INVENTION

Replicon-based vectors of positive strand RNA viruses have been developed for anti-viral and anti-cancer vaccines (reviewed in reference (25)). Several features make these vectors a desirable choice for development of highly efficient and safe vaccines. These include: (i) high level of expression of encoded heterologous genes (HGs) due to the ability of replicon RNA to amplify itself, (ii) exclusively cytoplasmic replication which eliminates any possible complications associated with nuclear splicing and/or chromosomal integration, (iii) inability of the replicon RNA to escape from transfected (or infected) cell thus limiting the spread of the vaccine vector in the immunized subject which makes these vectors biologically safe, and (iv) relatively small genome size (7-9 kb) allowing easy manipulations with their cDNA and generation of recombinants.

Replicon-based expression vectors have been developed for representatives of most positive strand RNA virus families, including alphaviruses, picomaviruses, and flaviviruses (reviewed in reference 25). However, the great majority of the data on immunogenic properties of replicon vectors in laboratory animals has been accumulated using replicons of alphaviruses such as Sindbis virus (SIN), Semliki Forest virus (SFV), and Venezuelan equine encephalitis virus (VEE) (see reference 47 for review and references 8, 10, 11, 39 for more recent studies). In general, these studies showed that alphavirus replicon vectors induced strong antibody and cytotoxic T lymphocyte (CTL) responses to encoded immunogens, and in most cases protected immunized animals from appropriate virus or tumour challenges. All three delivery modalities were effective, however VLP delivery was shown to be the most efficient. In comparative studies between conventional (non-replicating) plasmid DNA vectors and alphavirus DNA-based replicon vectors, the latter generally induced stronger immune responses and at significantly lower DNA concentrations (4, 17). However, in some studies the efficiency of immune responses induced by DNA-based alphavirus replicons appeared to depend on the nature of encoded immunogen, and the immunity to some immunogens was shown to be similar or lower than that induced by the same amount of conventional plasmid DNA vector (10, 23).

Replicon vectors derived from the flavivirus Kunjin (KUN) have been described in several publications (26, 28, 52, 53; International Publication WO 99/28487). The KUN replicon expression system, like alphavirus replicon systems, allows delivery of replicon RNA by three different modalities, i.e. as naked RNA, as VLPs, and as plasmid DNA (FIG. 1A) (25, 52, 53). Unlike homologous alphavirus replicon packaging systems (6, 33, 41), the KUN replicon packaging system makes use of a replicon expression vector derived from the unrelated SFV virus for production of KUN structural proteins (28, 52). This eliminates any potential problems associated with contamination of VLPs with infectious recombinant material. Additionally, in contrast to highly cytopathic alphavirus replicon vectors commonly used in immunization studies, KUN replicons appear to be noncytopathic and allowed prolonged expression of HGs both in vitro and in vivo (53). Although non-cytopathic alphavirus replicon vectors have been developed (1, 12, 40), no data on their immunogenic properties have been reported. In previous studies immunizations of mice with VLPs comprising a noncytopathic DNA-based KUN replicon vector expressing the P-galactosidase gene resulted in induction of an anti-β-galactosidase antibody responses (53; International Publication WO 99/28487). However, there has been no demonstration that KUN replicon vector systems can deliver immunogens capable of inducing protective immune responses.

### OBJECT OF THE INVENTION

The present inventors herein describe the first, flavivirus replicon-based vaccine vaccine delivery system that can provide long-term, protective immunity by way of antibody- and T cell-mediated responses.

Furthermore, the present inventors provide modifications and improvements to flaviviral replicons and to packaging systems useful in VLP production.

It is therefore an object of the invention to provide an improved flavivirus replicon vector system useful in vaccine delivery.

### SUMMARY OF THE INVENTION

The invention is therefore broadly directed to a flaviviral expression system and flaviviral replicon, expression vector and expression construct useful in same. In a particular embodiment, the invention is directed to a flaviviral vaccine delivery system.

In one aspect the invention provides an expression vector comprising:

(i) a nucleotide sequence encoding a flavivirus replicon that is incapable of producing infectious virus wherein said flavivirus replicon encodes one or more mutated flaviviral non-structural proteins;

(ii) an insertion site for a heterologous nucleic acid;

(iii) a promoter operably linked to the nucleotide sequence encoding the flavivirus replicon; and

(iv) at least one autoprotease-encoding nucleotide sequence.

Suitably, a flavivirus replicon encoding the mutated flaviviral nonstructural protein (s) allows more efficient establishment of persistent replication in an animal cell compared to a flavivirus replicon encoding corresponding wild-type protein(s).

Preferably, said mutated flaviviral non-structural protein in (i) is selected from the group consisting of:

(a) a mutated nonstructural protein NS1;

(b) a mutated nonstructural protein NS2A; and

(c) a mutated nonstructural protein NS5.

More preferably, said mutated flaviviral non-structural protein in (i) is selected from the group consisting of:

(i) a nonstructural protein NS1 having a mutation of Proline 250 to Leucine;

(ii) a nonstructural protein NS2A having a mutation of Alanine 30 to Proline;

(iii) a nonstructural protein NS2A having a mutation of Asparagine 101 to Aspartate; and

(iv) a nonstructural protein NS5 having a mutation of Proline 270 to Serine.

In a preferred embodiment, the or each autoprotease-encoding nucleotide sequence encodes a foot and mouth disease virus 2A autoprotease.

In one particular embodiment, the expression vector comprises one autoprotease-encoding nucleotide sequence. Examples of this embodiment are designated SP6KUNrep5 and pKUNrep5.

In another particular embodiment, the expression vector comprises two autoprotease-encoding nucleotide sequence, wherein a first of said at least two autoprotease-encoding nucleotide sequences is located 5′ of said insertion site and a second of said at least two autoprotease-encoding nucleotide sequences is located 3′ of said insertion site. Examples of this embodiment are designated SP6KUNrep6 and pKUNrep6.

In another aspect the invention provides an expression construct comprising:

(i) a nucleotide sequence encoding a flavivirus replicon that is incapable of producing infectious virus wherein said flavivirus replicon encodes one or more mutated flaviviral non-structural proteins;

(ii) a heterologous nucleic acid;

(iii) a promoter operably linked to the nucleotide sequence encoding the flavivirus replicon; and

(iv) at least one autoprotease-encoding nucleotide sequence.

Suitably, a flavivirus replicon encoding the mutated flaviviral nonstructural protein (s) allows more efficient establishment of persistent replication in an animal cell compared to a flavivirus replicon encoding corresponding wild-type protein(s).

Preferably, said mutated flaviviral non-structural protein in (i) is selected from the group consisting of:

(a) a mutated nonstructural protein NS1;

(b) a mutated nonstructural protein NS2A; and

(c) a mutated nonstructural protein NS5.

More preferably, said mutated flaviviral non-structural protein in (i) is selected from the group consisting of:

(i). a nonstructural protein NS1 having a mutation of Proline 250 to Leucine;

(ii) a nonstructural protein NS2A having a mutation of Alanine 30 to Proline;

(iii) a nonstructural protein NS2A having a mutation of Asparagine 101 to Aspartate; and

(iv) a nonstructural protein NS5 having a mutation of Proline 270 to Serine.

In a preferred embodiment, the or each autoprotease-encoding nucleotide sequence encodes a foot and mouth disease virus 2A autoprotease.

In one embodiment, said expression construct comprises at least two autoprotease-encoding nucleotide sequences, wherein a first of said at least one autoprotease-encoding nucleotide sequences is located 5′ of said heterologous nucleic acid and a second of said at least one autoprotease-encoding nucleotide sequences is located 3′ of said heterologous nucleic acid.

In a particular embodiment, this aspect provides an RNA that is transcribable from a DNA expression construct wherein the RNA comprises:

(i) an RNA sequence encoding a flavivirus replicon that is incapable of producing infectious virus wherein said flavivirus replicon encodes one or more mutated flaviviral non-structural proteins; and

(ii) a heterologous RNA sequence encoding a heterologous protein..

In one embodiment, the promoter is operable to promote RNA transcription in vivo.

More preferably, the promoter is operable to promote RNA transcription in a mammalian cell.

An example of a preferred promoter operable in a mammalian cell is a CMV promoter.

In another embodiment, the promoter is operable to promote RNA transcription in vitro.

An example of a preferred promoter operable to promote RNA transcription in vitro is an SP6 promoter.

In particular embodiments, non-limiting examples of expression constructs of the invention include RNALeuMpt, RNAProMpt, KUNRNAgag comprising an SP6 promoter for in vitro RNA expression or DNALeuMpt, DNAProMpt, KUNDNAgag comprising a CMV promoter for intracellular RNA expression.

In yet another aspect, the invention provides an expression system comprising:

(i) the expression vector of the first-mentioned aspect or the expression construct of the second-mentioned aspect; and

(ii) at least another expression construct that is capable of expressing one or more proteins that facilitate packaging of said expression vector or construct into flavivirus virus like particles (VLPs).

In particular embodiments, said another expression construct is a packaging construct selected from the group consisting of: SFVMEC/L713P, SFVMEC/L713P/Neo and pSFV3L713PLacZNeo. These constructs include a substitution of proline for leucine 713 in the SFV nsP2 gene to decrease cytopathicity. SFVMEC/L713P/Neo and pSFV3L713PLacZNeo are particularly suited to generation of stable cell lines.

In still yet another aspect, the invention provides a pharmaceutical composition comprising an RNA that is transcribable from a flaviviral DNA expression construct wherein the RNA encodes:

(i) a flavivirus replicon that is incapable of producing infectious virus; and

(ii) an immunogenic protein.

In a further aspect, the invention provides a pharmaceutical composition comprising a flaviviral DNA expression construct from which RNA is transcribable in an animal cell, wherein the transcribable RNA encodes:

(i) a flavivirus replicon that is incapable of producing infectious virus; and

(ii) an immunogenic protein.

Preferably, according to the aforementioned nucleic acid compositions, the DNA and RNA expression constructs include a nucleotide sequence that encodes at least one foot and mouth disease virus 2A autoprotease.

More preferably, the DNA and RNA constructs include a nucleotide sequence located 5′ of said heterologous nucleic acid that encodes a first said foot and mouth disease virus 2A autoprotease and a nucleotide sequence located 3′ of said heterologous DNA that encodes a second said foot and mouth disease virus 2A autoprotease.

In a still further aspect, the invention provides a pharmaceutical composition comprising one or more VLPs produced by the expression system of the third-mentioned aspect.

Preferably, the pharmaceutical compositions of the invention are immunotherapeutic compositions or more preferably, vaccines.

In a still yet further aspect, the invention provides methods of immunizing an animal including the step of administering the pharmaceutical composition of any of the aforementioned aspects to said animal to induce immunity in said animal.

Animals include humans, domestic livestock, companion animals, poultry and other animals of commercial importance, although without limitation thereto.

Preferably, the animal is a mammal.

More preferably, the animal is a human.

Immunity may be antibody-mediated and/or cell mediated immunity such as T cell mediated immunity.

In one embodiment, T cell immunity is characterized by a CD8+cytotoxic T lymphocyte (CTL) response.

Preferably, T cell immunity is characterized by induction of a long-term effector CD8+CTL response.

In another embodiment, T cell immunity is characterized by a CD4+T cell response.

In one particular embodiment, the method of immunization induces immunity to viral infection.

In another particular embodiment, the method of immunization induces immunity to cancers such as melanoma.

Throughout this specification, unless otherwise indicated, “comprise”, “comprises” and “comprising” are used inclusively rather than exclusively, so that a stated integer or group of integers may include one or more other non-stated integers or groups of integers.

### BRIEF DESCRIPTION OF THE FIGURES AND TABLES

Table 1. Sequential transfections of Kunjin and SFV replicon RNAs.

Table 2. Simultaneous transfections of Kunjin and noncytopathic SFV replicon RNAs.

Table 3. Production of secreted VPLs in SFVMECA12 cell line transfected with Kunjin replicon RNAs.

Table. 4. Effect of the incubation temperature on VLP production in SFVMECA12 cell line transfected with KUN-GAG replicon RNA.

Table. 5. VLP production in cell lines expressing noncytopathic SFV replicon and transfected with pSFVHelperCprME and KUN-GAG RNAs.

Table 6. Protection against challenge with rVVgag following immunization with KUNgag VLPs.

Table 7. Adaptive mutations in puromycin resistant BHK-KUN replicon cell clones No 11 and No 20

FIG. 1. Schematic representation of the replicon gene expression and delivery systems based on KUN replicon (A) and of the KUN replicon constructs encoding murine polyepitope (Mpt) (B). (A) KUN replicon RNA can be transcribed in vitro from plasmid DNA incorporating bacteriophage SP6 promoter and delivered as naked RNA via transfection or injection (26, 52). It can also be synthesized in vivo by cellular RNA polymerase II from transfected or injected plasmid DNA incorporating cytomegalovirus (CMV) promoter (53). In addition, KUN replicon RNA can be first packaged into virus-like particles (VLPS) using packaging system described previously (28) and then delivered by infection (52). Regardless of the delivery mode, once in the cytoplasm, KUN replicon RNA initiates self-replication leading to production of multiple RNA copies, translation of which results in enhanced production of encoded heterologous gene (HG) products. CAP represent cap structure added to the 5′-terminus of replicon RNA molecules either synthetically during in vitro transcription or naturally during in vivo transcription or KUN RNA replication, to ensure efficient initiation of translation. (B) KUN replicon constructs contain sequences required for KUN RNA replication, i.e. 5′ and 3′ untranslated regions (UTRs), sequences coding for the first 20 amino acids of KUN C protein (C20) and the last 22 amino acids of KUN E protein (E22), and the entire nonstructural region coding for the KUN nonstructural proteins NS1 (shown as NS1), NS2A, NS2B, NS3, NS4A, NS4B, and NS5 (shown as NS2-NS5). In addition, constructs contain either SP6 or CMV promoters upstream of the KUN 5′UTR to drive in vitro or in vivo RNA transcription, respectively, and the antigenomic sequence of the hepatitis delta virus ribozyme (HDVr) and polyadenylation signal from simian virus 40 (pA) inserted downstream of the 3′UTR to ensure production of KUN replicon RNA molecules with precise 3′termini for efficient initiation of replication. To allow cytoplasmic release of Mpt peptide from the KUN C20 and E22 peptides, the constructs contain two copies of 2A autoprotease of the foot and mouth disease virus (FMDV2A), one upstream and another downstream of the Mpt sequence. Pro and Leu variants contain amino acids Pro or Leu at the position 250 in the KUN NS1 gene, respectively.

FIG. 2. Evidence of replication of KUN replicon constructs encoding murine polytope in transfected BHK21 cells. (A) IF analysis of BHK21 cells transfected with DNA-based or RNA-based KUN replicons encoding Mpt. Cells on coverslips were transfected with KUN replicon-polytope DNAs (DNALeuMpt and DNAProMpt) or RNAs (RNALeuMpt and RNAProMpt) and assayed for expression of KUN NS3 proteins by IF at 48 h after transfection. (B) Northern blot of total RNA from BHK21 cells transfected with KUN replicon-polytope DNAs and RNAs. Transfection of cells, RNA isolation and Northern was performed as described in Materials and Methods. Left panel shows results of Northern blot with KUN-specific probe representing KUN 3′ untranslated region, right panel shows results of Northern blot of the same membrane rehybridized with the probe representing Mpt sequence. Samples from DNA-transfected cells contain 6 μg of total RNA and samples from RNA-transfected cells contain 12 μg of total RNA. pKUNβRep2(dGDD) lines show RNA samples isolated from BHK cells transfected with pKUNβRep2(dGDD) DNA producing non-replicating KUN replicon RNA encoding β-galactosidaze gene (53). The position of this RNA in the left panel is indicated by the asterisk indicated by the asterisk. Control RNA represent 10 ng of in vitro transcribed RNALeuMpt RNA.

FIG. 3. CTL responses specific for YPHFMPTML (YPH), RPQASGVYM (RPQ), TYQRTRALV (TYQ) and SYIPSAEKI (SYI) epitopes induced by immunization with various KUN replicon-based vectors encoding Mpt immunogen. (A) ELISPOT analysis of Balb/c (n=4 per group) splenocytes following immunization with the indicated vaccines. (B) ⁵¹Chromium (Cr) release assay of restimulated splenocyte populations from Balb/c mice (n=3 per group) immunized with VLPProMpt. A standard 6 h Cr release assay was performed using labeled target cells sensitized with (filled squares) or without (empty squares) the indicated peptides.

FIG. 4. CD8+CTL responses specific for SYIPSAEKI epitope induced after immunization with different doses of DNA-based KUN replicons encoding the Mpt. DNALeuMpt and DNAProMpt were serially diluted in PBS and the indicated doses injected into quadriceps muscles of Balb/c mice (n=4 per group). Mice were sacrificed 2 weeks later and CD8+CTL responses measured by ELISPOT assay.

FIG. 5A & B. Recombinant vaccinia virus and B16-OVA tumour challenge following RNALeuMpt vaccination. (A) Recombinant vaccinia challenge. Balb/c mice (n=6 per group) were vaccinated once with RNALeuMpt or RNALeuControl RNAs and then challenged with rVVMpt Vaccinia virus titres in ovaries were measured at day 4 after infection. (B) B16-OVA challenge. Balb/c mice (n=6 per group) were immunized twice with either RNALeuControl or RNALeuMpt RNAs or once with recombinant vaccinia encoding murine polytope (rVVMpt) and were then challenged with B16-OVA cells. The top graph shows the average tumour area for 12 tumour sites for each of the three groups of mice. The tumour growth lines stop on the date when the first tumour in a group reached the size of 15×15 mm requiring the animal's euthanisation. The bottom graph represents Kaplin-Meier survival curves of the three groups of mice. Mice were euthanasied when the tumour size reached 15×15 mm.

FIG. 5C. Kaplan-Meier survival curves of B16-OVA tumour challenge following VLPLeuMpt vaccination. Mice were sacrificed when the tumour size reached 15×15 mm. Log rank statistic p<0.01, VLPLeuMpt compared to rVV.

FIG. 6. Examples of expression vectors pKUNrep4 (previously published in reference 53), pKUNrep5 and SP6KUNrep5 containing one copy of FMDV2A protease and EMCV IRES, SP6KUNrep6 and pKUNrep6, containing two copies of FMDV 2A protease. pKUNrep5 was made by deleting the PAC gene from pKUNrep4. SP6KUNrep5 was prepared by replacing CMV promoter with SP6 promoter in the pKUNrep5 plasmid. pKUNrep5 and SP6KUNrep5 vectors have only one FMDV2A sequence for cleavage to release a heterologous gene product with a near authentic N-terminus. The EMCV IRES sequence downstream of the inserted heterologous gene allows independent initiation of translation of KUN nonstructural genes, so the heterologous gene can have a stop codon and thus an authentic C-terminus as well. pKUNrep6 vector forms the basis of theDNALeuMpt, and DNAProMpt expression construct, and SP6KUNrep6 forms the basis of the RNALeuMpt and RNAProMpt expression constructs, as shown in FIG. 1B.

FIG. 7. Construction of pSFVMECL713P. Initially the SacI-XhoI fragment from pSFV1 vector (Life Technologies), containing the Semliki Forest Virus nsP2 gene, was ligated into pBluescriptII KS for introduction of a mutation at amino acid 713. This was performed using overlapping PCR which generated an AvrII site into the nsP2 gene, modifying the amino acid 713 from a leucine to proline. This mutation, L713P, should produce a noncytopathic strain of SFV (59). The resulting plasmid was subsequently named pBSKS/SFVSac-Xho fit. The RsrII-Bsu36I fragment of this construct was then transferred into pSFVMEC105 to produce the construct, pSFVMECL713P.

FIG. 8. Construction of pSFVMECL713PNeo. The sequence for the EMCV internal ribosome entry site (IRES) and the neomycin gene were excised from pBS-CIN4IN using MluI and XbaI. The IRES/Neo genes were then inserted into the BamHI site of pSFVMECL713P downstream of the inserted Kunjin core sequence. This construct was then used to establish a stable cell line expressing KUN structural genes.

FIG. 9. Construction of pSFV3L713PLacZNeo. Initially, the IRES/Neo gene was inserted into pSFV3LacZ using the same MluI-XbaI end-filled fragment from pBS-CIN4IN as was used for the cloning of IRES/Neo into pSFVMECL713Pneo. This MluI-XbaI fragment was inserted into the SmaI site of pSFV3LacZ. Subsequently the L713P mutation, which confers a noncytopathic phenotype of SFV replicon, was introduced by transferring the SpeI-NotI fragment containing the SFV nonstructural proteins 14 from pSFVMECL713P to pSFV3LacZNeo using the same restriction sites.

FIG. 10. Construction of pSFVHelperprMEC. To construct a helper plasmid for expression of Kunjin structural genes in the pSFV3L713PIacZNeo stable cell line, the KUN prMEC gene cassette was excised from pSFVMEC105 using the MscI-SpeI sites. This fragment was then inserted into pSFVHelper2 (Life Technologies) using the same restriction sites and replaced the SFV structural proteins. It should be noted that in this construct prME and C are placed under control of two separate 26S promoters.

FIG. 11. Construction of pSFVHelperCprME. A Kunjin CprME cDNA fragment with BglII sites at the ends was generated by PCR using Pfu polymerase, appropriate primers and FLSDX plasmid DNA containing full-length Kunjin cDNA (27) as a template. This CprME fragment was then ligated with a fragment containing pSFVHelper2 vector which was also generated by PCR and contained BamHI sites at each end.

FIG. 12. Anti- HIV gag antibodies in mice responded to immunization with KUNgagVLPs (n=6 per group) and rVVgag (n=3 per group). Mice were immunized twice with 10⁶ PFU of KUNgagVLPs or control KUN VLPs (KUN Control), or once with 10⁶ PFU of rVVgag. Two to three weeks after the last immunization mouse sera were analysed for anti-gag antibodies using an ELISA against purified recombinant gag protein.

FIG. 13. T cell proliferation assay of splenocytes from mice immunized with KUNgagVLPs. HIV-1 gag protein-pulsed splenocytes were used to stimulate splenocytes. SE=standard error. A stimulation index can be calculated as counts with HIV-1 gag antigen (black bars)/counts without antigen (white bars) respectively immunized and non-immunized mice.

FIG. 14. Long term immune responses elicited by KUN replicon VLP vaccines. (A) ELISPOT response to AMQ epitope in BALB/c mice (n=4 per group) 2 weeks and 6 months after a single immunization with KUNgagVLP. (B) ELISPOT analysis of splenocytes from BALB/c mice (n=8 per group) detected at 6 months after two immunizations (separated by 4 weeks) with the KUN replicon VLPs encoding murine polytope immunogen (mpt). CD8+T cell responses are shown for individual epitopes encoded by Mpt, i.e. YPHFMPTML (YPH), RPQASGVYM (RPQ), TYQRTRALV (TYQ) and SYIPSAEKI (SYI). (C) BALB/c mice (n=6 per group) were vaccinated twice with KUNmpt VLP separated by 4 weeks, and 10 months after the second immunization mice were challenged with recombinant vaccinia virus encoding mpt (rVVmpt). Vaccinia virus titres in ovaries were measured at day 4 after infection.

FIG. 15. β-galactosidase expression from KUN replicon RNA in different puromycin resistant BHK cell clones at passage 4.

FIG. 16. Effect of adaptive mutations on the ability of KUN replicon RNA to establish persistent replication in BHK21 cells. From left to right the plates are repPACβ-gal, repPACβ-gal NS2A(A-P), repPACβ-gal NS2A(N-D) and repPAβ-gal NS5(P-S).

### DETAILED DESCRIPTION OF THE INVENTION

The present inventors describe immunization of mice with KUN replicons expressing the murine CTL polyepitope or polytope (Mpt) and H[V-1 gag as immunogens, and delivered in the form of naked RNA, VLPs, or plasmid DNA. All these modes of vaccination resulted in induction of CD8+CTL responses specific to encoded epitopes and in the case naked RNA and VLPs, protected mice from viral and tumour challenges. Furtherm immunization of mice with KUN replicons expressing the complete HIV-1 gag gene delivered as VLPs induced antibody and CD4+T cell responses specific to gag and protec mice from a challenge with recombinant vaccinia virus expressing the gag gene.

Also, the KlN replicon has been modified (compared to that described in 99/28487) by insertion of FMDV2A autoprotease sequences 5′ and 3′ of the sequence encoding the heterologous immunogen. This modification results in autoproteolytic cleav of the expressed fusion protein to liberate the protein immunogen substantially free extraneous amino acid sequence. Further modifications include use of a mutated nucleol sequence encoding one or more of an NS1, NS2A and NS5 protein component of flavivirus replicon of the invention, which mutation results in more efficient establishmen persistent replication. The present invention also provides a novel, SFV-based v packaging system that is also less cytopathic than the SFV-based system described International Publication WO 99/28487.

KUN Replicon Expression Vectors and Constructs

The term “nucleic acid” as used herein designates single-or double-stranded mRNA, RNA, cRNA and DNA inclusive of cDNA and genomic DNA.

As used herein, “flavivirus” and “flaviviral” refer to members of the family Flaviviridae within the genus Flavivirus, which contains 65 or more related viral species. Typically, flavivirus are small, enveloped RNA viruses (diameter about 45 nm) with peplomers comprising a single glycoprotein E. Other structural proteins are designated C (core) and M (membrane-like). The single stranded RNA is infectious and typically has a molecular weight of about 4×10⁶ with an m7G‘cap’ at the 5′ end but no poly(A) tract at the 3′ end; it functions as the sole messenger. Flaviviruses infect a wide range of vertebrates, and many are transmitted by arthropods such as ticks and mosquitoes, although a separate group of flaviviruses is designated as having no-known-vector (NKV).

Particular, non-limiting examples of flavivirus are West Nile virus, Kunjin virus, Yellow Fever virus, Japanese Encephalitis virus, Dengue virus, Montana Myotis leukoencephalitis virus, Usutu virus, and Alkhurma virus.

Although a preferred flaviviral replicon of the invention is derived from Kunjin virus, it will be appreciated by persons skilled in the art that the expression vector, expression construct and expression system of the invention may be practised using any flaviviral replicon.

Examples of flavivirus replicons that are relatively well characterized include West Nile Virus lineage 1 (ref. 61) and lineage II strain (ref. 62) replicons.

Flavivirus replicons contemplated by the present invention include any self-replicating component(s) derivable from flavivirus RNA as described for example in International Publication WO 99/28487.

As generally used herein, flavivirus replicons are derived from flavivirus or are otherwise of flavivirus origin. Thus, in the context of this specification “a nucleotide sequence encoding a flavivirus replicon” is a DNA or RNA sequence that comprises sequence information from a flavivirus replicon or at least a portion thereof sufficient for replication while being incapable of producing infectious virus.

For example, as will be understood by persons skilled in the art, DNA-based constructs of the invention referred to herein comprise a DNA copy of replicon RNA, which is complementary to or otherwise derived from said replicon RNA.

Suitably, the flavivirus replicon is replication competent while being “incapable of producing infectious virus”. By this is meant that the flavivirus replicon is unable to express one or more structural proteins either in their entirety or in part, that are required for viral packaging. A detailed description of modifications to Kunjin flaviviral replicons to disable viral packaging is provided in International Publication WO 99/28487.

In a preferred embodiment, the flavivirus replicon comprises:

(i) 5′ and 3′ untranslated (UTR) sequences and sequences encoding the first 20 amino acids of C protein (C20) and the last 22 amino acids of E protein (E22) respectively; and

(ii) nucleotide sequence encoding nonstructural proteins NS1, NS2A, NS2B, NS3, NS4A, NS4B and NS5.

In a particular aspect, one or more of said nonstructural proteins is mutated.

In one particular embodiment, Proline residue 250 of the NS1 protein is substituted by Leucine.

In another particular embodiment, Alanine 30 is substituted by Proline in the nonstructural protein NS2A.

In yet another particular embodiment, Asparagine 101 is substituted by Aspartate in the nonstructural protein NS2A.

In still yet another particular embodiment, Proline 270 is substituted by Serine in the nonstructural protein NS5.

It will also be appreciated that each of the above mutations or substitutions may be present individually or in combination in a flavivirus replicon of the invention.

According to the present invention an “expression vector” comprises the aforementioned flavivirus replicon together with one or more other regulatory nucleotide sequences. Such regulatory sequences include but are not limited to a promoter, internal ribosomal entry site (IRES), restriction enzyme site(s) for insertion of one or more heterelogous nucleic acid(s), polyadenylation sequences and other sequences such as an antigenomic sequence of the hepatitis delta virus ribozyme (HDVr) that ensure termination of transcription and precise cleavage of 3′ termini, respectively.

It will be appreciated that expression vector and expression construct of the invention include a nucleotide sequence encoding at least one autoprotease.

Preferably, said nucleotide sequences encodes at least one foot and mouth disease virus 2A autoprotease, or more preferably respective said nucleotide sequences each encode a respective foot and mouth disease virus 2A autoprotease.

Preferably, in use an expressed fusion protein comprises a heterologous protein flanked by an N-terminal foot and mouth disease virus 2A autoprotease and a C-terminal foot and mouth disease virus 2A autoprotease. This arrangement results in the foot and mouth disease virus 2A autoproteases cleaving the fusion protein to liberate the heterologous protein substantially free of other amino acid sequence(s).

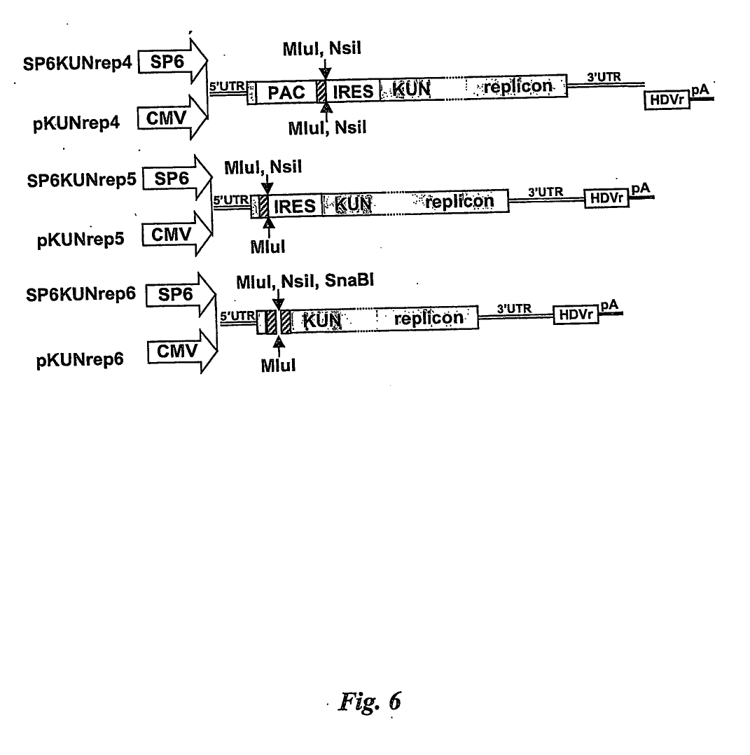

Particular examples of KUN replicon vectors of the invention are pKUNrep5 and SP6KUNrep5 encoding one FMDV2A protease and EMCV IRES and SP6KUNrep6 and pKUNrep6 which encode two FMDV2A proteases, as shown in FIG. 6.

According to the present invention, an “expression construct” is an expression vector into which a heterologous nucleic acid has been inserted so as to be expressible in the form of RNA and/or as an encoded protein

Said heterologous nucleic acid may encode one or more peptides or polypeptides, or encode a nucleotide sequence substantially identical or substantially complementary to a target sequence.

By “protein” is meant an amino acid polymer. Amino acids may include natural (ie genetically encoded), non-natural, D- and L- amino acids as are well known in the art.

A “peptide” is a protein having less than fifty (50) amino acids.

A “polypeptide” is a protein having fifty (50) or more amino acids.

Heterologous nucleic acids may encode proteins derived or obtained from pathogenic organisms such as viruses, fungi, bacteria, protozoa, invertebrates such as parasitic worms and arthropods or alternatively, may encode mutated, oncogenic or tumour proteins such as tumour antigens, derived or obtained from animals inclusive of animals and humans. Heterologous nucleic acids may also encode synthetic or artificial proteins such immunogenic epitopes constructed to induce immunity.

In one embodiment, said heterologous nucleic acid encodes one or more immunogenic peptides or polypeptides, although without limitation thereto.

Preferably, the one or more immunogenic peptides or polypeptides comprise one or more T cell epitopes.

In one particular embodiment, said heterologous nucleic acid encodes multiple peptide epitopes (polyepitope) such as a murine polyepitope (Mpt) to be described in more detail hereinafter. Examples of epitope sequences include YPHFMPTNL, RPQASGVYM, TYQRTRALOV, SYIPSAEKI and SIINFEKL but without limitation thereto.

In another particular embodiment, said heterologous nucleic acid encodes a HIV-1 gag gene or a fragment thereof.

In expression constructs of the invention, the promoter is operably linked or connected to said flavivirus replicon.

By “operably linked” or “operably connected” is meant that said promoter is positioned to initiate, regulate or otherwise control in vitro or in vivo transcription of RNA encoding said flavivirus replicon, said heterologous nucleic acid and other regulatory sequences that facilitate RNA processing and protein expression.

Preferably, the promoter is located 5′ of the flavivirus replicon.

A preferred promoter for in vitro transcription of RNA from said DNA expression construct is an SP6 promoter.

A preferred promoter for in vivo transcription of RNA from said DNA expression construct in mammalian cells is a cytomegalovirus (CMV) promoter.

However, it will be appreciated that other well-known promoters active in mammalian cells are contemplated, including an SV40 promoter, a human elongation factor alpha promoter and an alpha crystallin promoter, although without limitation thereto,

In embodiments where the expression construct of the invention is used to generate stably-transformed host cells, the expression vector further comprises a selectable marker gene to allow the selection of transformed host cells. Selectable marker genes are well known in the art and include neomycin transferase and puromycin N-acetyl transferase, without limitation thereto. The selectable marker genes can be inserted either in place of deleted structural genes or into the 3′UTR region.

Suitable host cells for protein expression may be any eukaryotic cell line that is competent to effect transcription, translation and any post-transcriptional and/or post-translational processing or modification required for protein expression. Examples of mammalian cells typically used for nucleic acid transfection and protein expression are COS, Vero, CV-1, BHK21, HEK293, Chinese Hamster Ovary (CHO) cells, NIH 3T3, Jurkat, WEHI 231, HeLa and B16 melanoma cells without limitation thereto.

Transfection of cells may be achieved by methods well known in the art such as calcium phosphate precipitation, electroporation, lipofectamine, lipofectin and other lipophilic agents, calcium phosphate precipitation, DEAE-Dextran, microparticle bombardment, microinjection and protoplast fusion.

Examples of expression constructs of the invention include RNALeuMpt, RNAProMpt, KUNRNAgag, DNALeuMpt, DNAProMpt and KUNDNAgag, as will be described in more detail hereinafter.

Viral Packaging

According to the third-mentioned aspect of the invention, there is provided a flaviviral expression system comprising:

(i) a flaviviral expression construct as hereinbefore described; and

(ii) at least another expression construct that is capable of expressing one or more proteins that facilitate packaging of said expression construct of the second- mentioned aspect into flavivirus virus like particles (VLPs).

Any non-flavivirus derived vector may be used to express the one or more structural proteins required for viral packaging and production of VLPs. For example, said another construct could be derived from another alpha virus such as semliki forest virus (SFV) or sindbis virus (SIN) or from DNA viruses such as adenovirus, fowlpox virus or vaccinia virus.

An example of said another construct (an SFV-derived construct) useful in production of VLPs and the preparation and purification of VLPs is provided in detail hereinafter.

In other embodiments, the present invention provides novel packaging systems that improve and simplify the packaging efficiency of the system described in International Publication WO 99/28487

It will be appreciated that in certain broad embodiments, flaviviral packaging may be achieved by:

(a) transient transfection of host cells (such as hereinbefore described) with a flaviviral expression construct and said another expression construct that provides structural proteins required for viral packaging;

(b) transient transfection of host cells with a flaviviral expression construct, wherein the host cells have been stably transfected with a packaging construct that provides structural proteins required for viral packaging.

With regard to (a), the expression constructs may be co-transfected or may be separately transfected within a time frame that allows optimal VLP production.

With regard to the above, “transected” is used for convenience as a general term encompassing transient or stable introduction of foreign genetic material into a host cell.

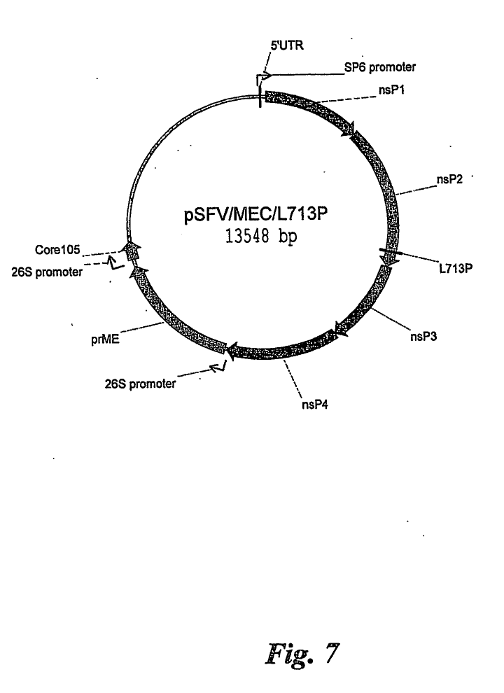

In one particular embodiment, modification of SFV-derived packaging construct SFV-MEC105 to decrease its cytopathicity was achieved by introducing mutation of amino acid 713 in the nsP2 gene from leucine to proline (SFVMEC/L713P; FIG. 7).

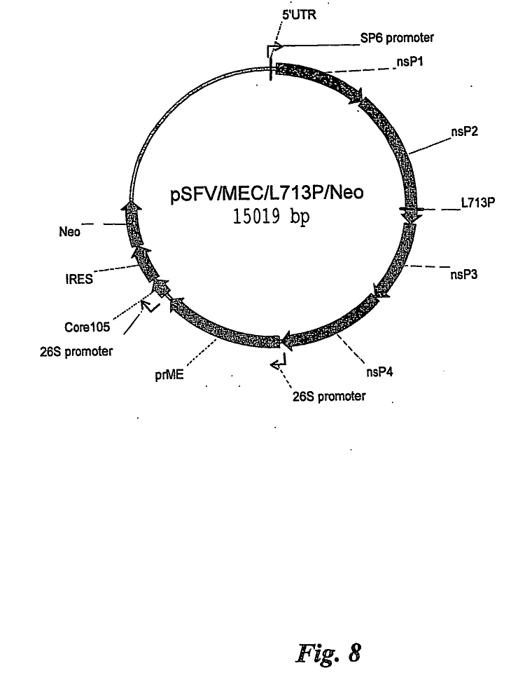

In another particular embodiment, the SFV-MEC/L713P construct includes an IRES-Neo cassette (SFVMEC/L713P/Neo; FIG. 8) to facilitate establishment of a stably expressing cell line by selection with antibiotic G418. Transfection of this cell line with Kunjin replicon RNAs allows its replication and the production of Kunjin VLPs.

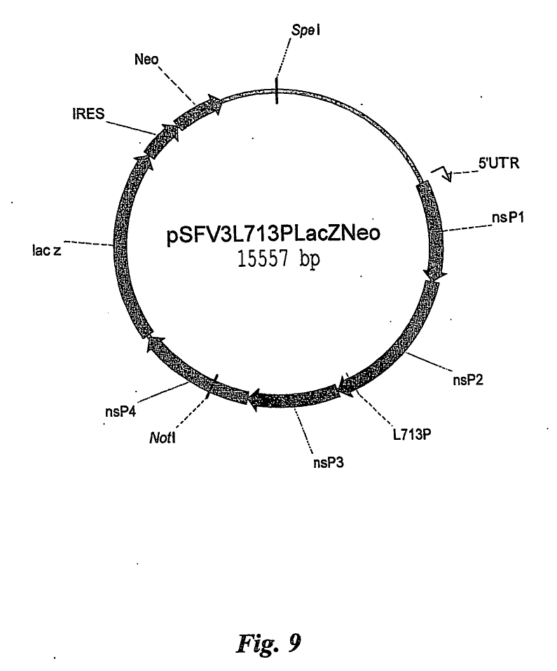

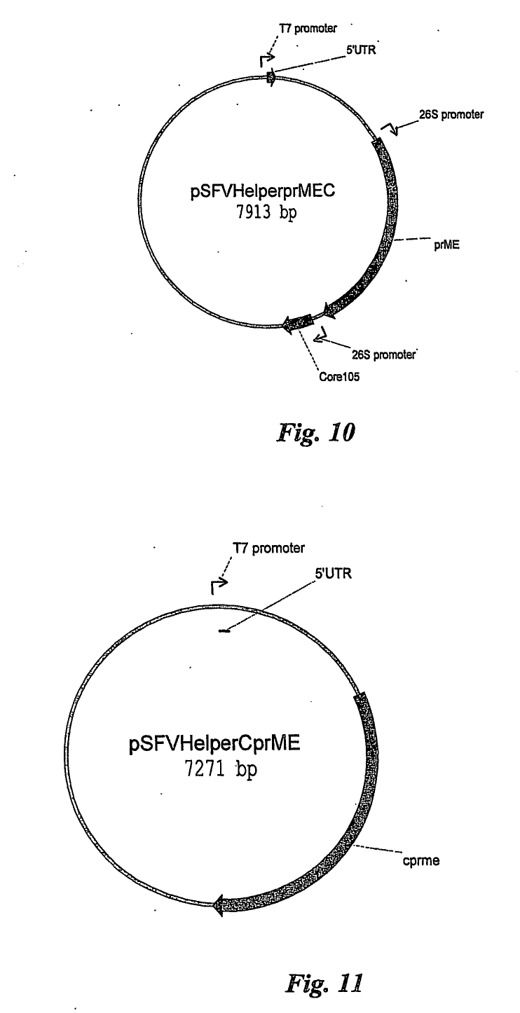

In yet another particular embodiment, a noncytopathic SFV replicon RNA construct pSFV3L713PLacZNeo (FIG. 9) is provided for amplification of transfected SFV helper RNAs pSFVHelperprMEC (FIG. 10) and pSFVHelperCprME (FIG. 11) to thereby express Kunjin structural proteins.

An example of said another construct (SFV-derived constructs), stable cell lines expressing said SFV-derived constructs, and different packaging protocols useful in production of VLPs and the preparation and purification of VLPs is provided in detail hereinafter.

Pharmaceutical and Immunotherapeutic Compositions and Vaccines

A particular aspect of the invention relates to use of the flaviviral expression construct and expression system as a vaccine delivery system. However, the invention more broadly provides a pharmaceutical composition not limited to use in vaccine delivery, but inclusive of immunotherapeutic compositions and vaccines that may comprise:

(i) VLPs produced by the expression system of the invention;

(ii) an RNA transcribed from a DNA expression construct of the invention; or

(iii) a plasmid DNA expression construct of the invention directing transcription of replicating RNA in vivo.

The pharmaceutical composition may further comprise a pharmaceutically-acceptable carrier, diluent or excipient

Such compositions may be delivered for the purposes of generating immunity, preferably protective immunity, to pathogens such as viruses, bacteria, protozoan parasites and invertebrate parasites although without limitation thereto.

In an alternative embodiment, immunoiherapeutic treatment of cancers, such as melanoma, is contemplated by the present invention.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, 1991) which is incorporated herein by reference.

Any safe route of administration may be employed for providing a patient with the composition of the invention. For example, oral, rectal, parenteral, sublingual, buccal, intravenous, intra-articular, intramuscular, intra-dermal, subcutaneous, inhalational, intraocular, intraperitoneal, intracerebroventricular, transdermal and the like may be employed. Intra-muscular and subcutaneous injection is appropriate, for example, for administration of immunotherapeutic compositions, proteinaceous vaccines and nucleic acid vaccines.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Pharmaceutical compositions of the present invention suitable for oral or parenteral administration may be presented as discrete units such as capsules, sachets or tablets each containing a predetermined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

The above compositions may be administered in a manner compatible with the dosage formulation, and in such amount as is pharmaceutically-effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

Immunotherapeutic compositions of the invention may be used to prophylactically or therapeutically immunize animals such as humans.

However, other animals are contemplated, preferably vertebrate animals including domestic animals such as livestock and companion animals.

As will be described in more detail hereinafter, vaccines of the present invention may be in the form of VLPs, RNA or DNA.

Immune responses may be induced against viruses, tumours, bacteria, protozoa and other invertebrate parasites by expressing appropriately immunogenic proteins and peptide epitopes inclusive of polyepitopes using the vaccine of the invention.

Preferably, the immune response involves induction of antibodies, CD8+CTLs and/or CD4+T cells.

More preferably, the immune response involves induction of long term effector CD8+CTLs.

In one particular embodiment, the present inventors have demonstrated that immunization with an RNA and VLP vaccine encoding an ovalbumin-derived CTL epitope protected mice against challenge with B16 melanoma cells expressing ovalbumin.

In another particular embodiment, the present inventors have demonstrated that immunization with VLP vaccine encoding complete HIV-1 gag gene protected mice from the challenge with recombinant vaccinia virus expressing HIV-1 gag gene.

It will also be appreciated that immunotherapeutic compositions and vaccines of the invention may, in certain embodiments, include an adjuvant.

As will be understood in the art, an “adjuvant” means one or more substances that enhances the immunogenicity and/or efficacy of a vaccine composition. Non-limiting examples of suitable adjuvants include squalane and squalene (or other oils of animal origin); block copolymers; detergents such as Tween®-80; Quil® A, mineral oils such as Drakeol or Marcol, vegetable oils such as peanut oil; Corynebacterium-derived adjuvants such as Corynebacterium parvum; Propionibacterium-derived adjuvants such as Propionibacterium acne; Mycobacterium bovis (Bacille Calmette and Guerin or BCG); interleukins such as interleukin 2 and interleukin 12; monokines such as interleukin 1; tumour necrosis factor; interferons such as gamma interferon; combinations such as saponin-aluminium hydroxide or Quil-A aluminium hydroxide; liposomes; ISCOM® and ISCOMATRIX® adjuvant; mycobacterial cell wall extract; synthetic glycopeptides such as muramyl dipeptides or other derivatives; Avridine; Lipid A derivatives; dextran sulfate; DEAE-Dextran or with aluminium phosphate; carboxypolymethylene such as Carbopol'EMA; acrylic copolymer emulsions such as Neocryl A640 (e.g. U.S. Pat. No. 5,047,238); vaccinia or animal poxvirus proteins; sub-viral particle adjuvants such as cholera toxin, or mixtures thereof.

So that the invention may be readily understood and put into practical effect, the skilled person is directed the following non-limiting examples.

### EXAMPLE 1

### Materials and Methods

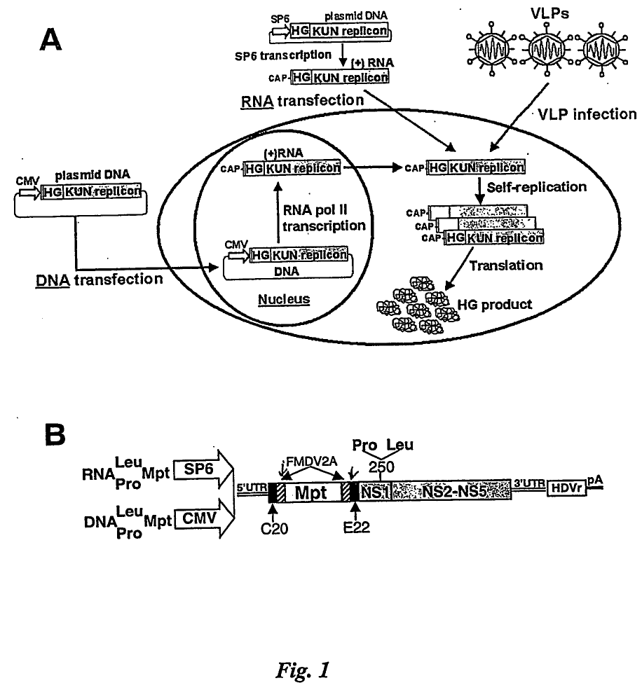

Plasmids. RNA-based (RNALeu) and DNA-based (DNALeu) KUN replicon vectors contain two copies of 2A autoprotease of the foot-and-mouse disease virus (FMDV2A), one upstream and another downstream of the cloning site, as well as leucine (Leu) at the amino acid position 250 in the KUN NS1 gene (FIG. 1B). RNAPro and DNAPro vectors contain proline (Pro) instead of Leu at the amino acid position 250 and they were constructed by replacing the SphI-SphI fragment spanning the entire NS1 gene in the RNALeu and DNALeu vectors with the corresponding SphI-SphI fragment from the KUN full-length cDNA plasmid 250pro (16). The murine polyepitope (Mpt) sequence was PCR amplified from the plasmid pSTMPDV (50) using primers Mpt-F (5′ GCGACGCGTCTAGAGCCAGCAACGAGAA-3′) and Mpt-R (5′-GTAACGCGTCTAAGTCCTCGGGGCCGG-3′). The PCR product was then digested with MluI and cloned into the MluI site of each of the four vectors, to produce plasmids RNALeuMpt, RNAProMpt, DNALeuMpt, and DNAProMpt, respectively (FIG. 1B).

DNA and RNA transfections, IF and Northern blot analyses. For DNA transfections, BHK21 cells in 6-well plates or on coverslips were tansfected with 2 or 0.4 μg plasmid DNAs, respectively, using Lipofectamine Plus transfection reagent (Life Technologies, Melbourne, Australia), as described by the manufacturer. RNAs were transcribed in vitro by the SP6 RNA polymerase from the XoI-linearised RNA-based plasmid DNAs and electroporated into BHK21 cells as described previously (26). Coverslips with transfected cells were fixed in cold acetone at 48 h after transfections, and assayed for expression of KUN NS3 protein by indirect immunofluorescence (IF) with anti-NS3 antibodies as described previously (56). Northern blot analysis of total RNA from transfected cells was performed as described previously (26) using ³²P-labelled cDNA probes representing either the KUN 3′UTR region or the Mpt sequence.

Preparation of VLPs. VLPs (VLPProMpt and VLPLeuMPT) were prepared as described previously (28, 52). Briefly, 2×10⁶ BHK21 cells were electroporated with ˜10-20 μg of in vitro transcribed RNAProMpt or RNALeuMPt RNA at 1.5 kV, 25 μF, ∝ resistance, two pulses at 10 sec interval. After electroporation cells were diluted in 8 ml DMEM/10% FCS and cultured in 60 mm dishes at 37° C. in CO₂ incubator. After 32 h cells were trypsinized and used for a second electroporation with in vitro transcribed SFV replicon RNA expressing KUN structural proteins (SFV-MEC105) (28) under the same conditions. At 18 h after the second electroporation, the media was replaced with 3 ml of DMEM/2% FCS and cells were incubated for a further 22 h. The media was harvested and clarified by spinning at 8000×g prior to storage in 1 ml aliquots at ˜70° C. The titre of infectious VLPs was determined by infection of BHK21 cells with 10-fold serial dilutions of the VLPs and counting the number of NS3-positive cells after IF analysis at 3040 h post-infection.

Immunization of mice. Female BALB/c (H-2^{d}) mice (6-8 weeks) were supplied by the Animal Resources Centre (Perth, Western Australia). Mice were immunized with the following formulations: (i) KUN replicon polytope DNA plasmids, DNALeuMpt and DNAProMpt encoding KUN replicons expressing Mpt and injected into the quadriceps muscles (100 μg in 100 μl PBS, im., 50 μl into each leg); (ii) in vitro transcribed KUN replicon RNAs, RNALeuMpt and RNAProMpt encoding Mpt were dissolved in DEPC-treated PBS and injected as above (˜30 μg in 100 μl, i.m., 50 μl in each leg); (iii) replicon RNA RNAProMPt or RNALeuMPT packaged into VLPs (VLPProMpt and VLPLeuMpt, respectively) was delivered in DMEM/2%FCS and injected i.p.(˜5×10⁵-10⁶ VLPs in 1 ml); (iv) a conventional plasmid DNA vaccine, pSTMPDV encoding Mpt (100 μg in 100 μl PBS, i.m., 50 μl into each quadriceps muscle) (50); and (v) a recombinant vaccinia virus encoding Mpt (10⁷ pfu in 200 μl RPMI 1640, i.p.) as described previously (51).

CTL assays. Epitope-specific IFNy secreting cells were enumerated by an enzyme linked immuno-spot (ELISPOT) assay using minimal CTL peptide epitopes as described previously (31). Briefly, flat-bottomed 96-well MultiScreen-HA cellulose ester membrane microtitre plates (Millipore Australia Ltd., North Ryde, Australia) were coated overnight with 5 μg/mL of rat anti-mouse IFNy antibody (clone RA-6A2, BD PharMingen, San Diego, USA). Coated plates were then blocked with 1% bovine serum albumin in PBS for 1 h at room temperature and washed three times with PBS containing 0.05% Tween 20 (Sigma). Splenocytes (1×10⁶/well) were plated in the first wells of the ELISPOT plate and serially diluted two fold. Recombinant human IL-2 (kindly provided by Cetus Corp., Emeryville, Calif., USA) (100 IU/ml) was added with peptide (Mimotopes, Clayton, Victoria, Australia) (1 μg/ml) and the plates incubated for 18 h. The cells were lysed, the plates were washed and IFNγ spots were detected by incubation first with biotinylated anti-mouse IFNγ antibody (clone XMG 1.2) (BD PharMingen) and then with streptavidin-alkaline phosphatase (BD PharMingen) and Sigma Fast BCIP/NBT substrate (Sigma). Spots were counted using a KS ELISPOT reader (Carl Zeiss Vision GmbH, Hallbergmoos, Germany).

⁵¹Chromium (Cr) release assays were performed as described previously (9). Briefly, mice were sacrificed 2-3 weeks post immunization with VLPProMpt and splenocytes were restimulated in vitro for 6 days by the addition of 1 jig/ml of peptide (Mimotopes). The resulting effector populations were split and equal numbers used in duplicate against peptide sensitized and unsensitised ⁵¹Cr-labeled P815 targets cells at the indicated effector to target ratios.

Vaccinia virus protection assay. Groups (n=6) of 6-8 week old female BALB/c mice were immunized once with ˜30 μg of RNALeuMpt or control (RNALeuControl) RNAs and after 3 weeks challenged i.p. with 10⁷ pfu per mouse of recombinant vaccinia virus encoding the murine polytope (rVVMpt) (51). At day 4 post-infection, both ovaries were removed, washed and homogenized in 1 ml of PBS using motorized grinder. 100 μl of ovary suspension was then mixed with 900 μl of PBS containing trypsin (Sigma) at 1 mg/ml and incubated for 30 mins at 37° C. The solution was briefly centrifuged at 10,000×g to remove debris and 100 μl of the supernatant were mixed with 900 μl of RPMI/10%FCS medium. The vaccinia virus titres in ovaries were then determined by plaque assay on confluent CV1 cells. The significance of the differences between the virus titres in the experimental and the control groups was calculated using Wilcoxan rank-sum test (7).

Tumour protection assay. B16 melanoma cells expressing ovalbumin (B16-OVA) (3) were kindly supplied by Dr K. Rock (Dana-Farber Cancer Institute, Boston, USA). Groups of C57BL/6 mice (n=6) were vaccinated i.m. twice separated by 4 weeks with ˜30 μg of either RNALeuMpt or control RNALeuControl RNAs, or once with rVVMpt, and 2 weeks after the last immunization mice were injected s.c. at two different sites on the back with B16-OVA cells (10⁵ cells per mouse). Hair around the injection site was removed with an electric shaver prior to injection to facilitate measurement of the emerging tumours. Growing tumours were monitored and the animals were sacrificed when tumour size reached 15 mm×15 mm. Mean tumour area was calculated for each treatment group at the indicated days after the tumour challenge. The difference in the mean tumour growth between groups was calculated using analysis of variance (45). The time when animals were sacrificed was also noted and represented as Kaplan-Meier survival curves (20). Differences between Kaplan-Meier survival curves were calculated using the log-rank statistic (20).

For tumour protection studies with VLPLeuMpt, female C5713L6 (H-2^{b}) mice (6-8 weeks old; n=6) were immunized intraperitoneally (ip) with these VLPs once (VLPLeuMptx1) or twice (VLPLeuMpt x 2, separated by two weeks); control VLP (VLPLeucontrol); or (iv) recombinant vaccinia virus encoding Mpt (rVVMpt). Two weeks after the last imunization, mice were challenged with 5×10⁵ B16-OVA melanoma tumour cells injected subcutaneously at two different sites.

### Results

Efficient Replication of KUN Replicons Encoding the Murine Polyepitope in Transfected Cells.

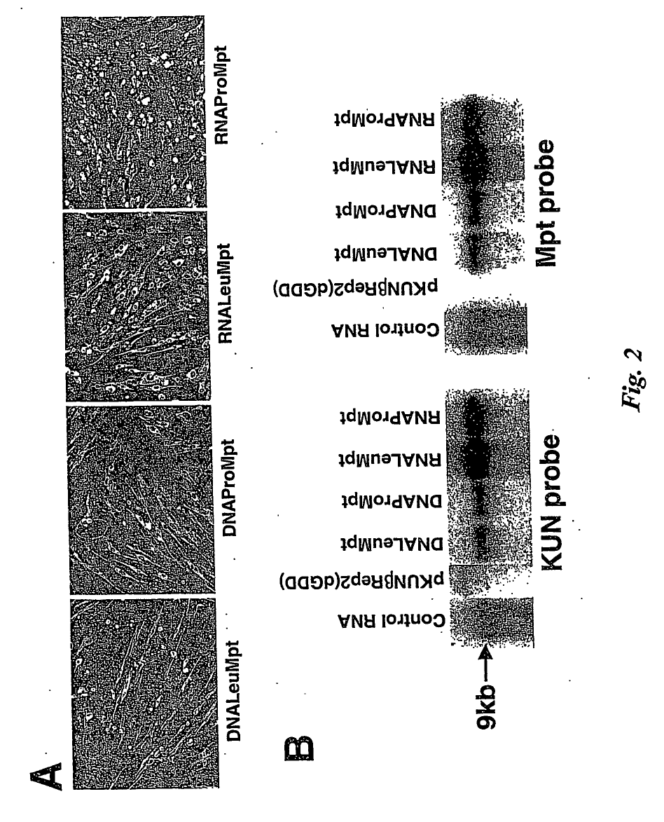

Plasmid DNAs DNALeuMpt and DNAProMpt encoding KUN replicon cDNAs expressing murine polytope (Mpt) were transfected into BHK21 cells and replication and expression of corresponding replicon RNAs transcribed in cells by RNA polymerase II were examined by Northern blot and IF analyses. In vitro synthesized Mpt-encoding KUN replicon RNAs RNALeuMpt and RNAProMpt were transfected into BHK21 cells by electroporation and analyzed for their replication and expression. IF analysis of transfected cells using KUN anti-NS3 antibodies showed that -50% cells were positive after transfection with DNA-based replicons, and ˜80% cells were positive after electroporation with RNA-based replicons (FIG. 2A). In previous experiments with electroporation of KUN replicon RNAs, expression of KUN NS3 could only be detected by IF when these RNAs were capable of replication (26, 27). Transfection of plasmid DNA encoding KUN cDNA, did however result in detection of expression of KUN proteins by IF, even when the KUN RNA was unable to replicate (30). To ensure that replicon RNAs produced in cells from transfected plasmid DNAs DNALeuMpt and DNAProMpt were replicating, we analysed total cellular RNA by Northern blot analysis with KUN-specific radiolabeled cDNA probe (FIG. 2A). Comparison of the intensity of the radioactive signal in these samples with that detected in the RNA sample isolated from cells transfected with the same amount of the control plasmid DNA pKUNβrep2(dGDD) producing replication-deficient KUN replicon RNA (53), showed that ˜5-10-fold higher amounts of KUN-specific RNAs were produced in DNALeuMpt and DNAProMpt-transfected cells than in pKUNβrep2(dGDD)-transfected cells. Previous studies with replication-competent and replication deficient KUN DNA-based replicon constructs showed similar differences in the amounts of accumulated RNAs (53). These results demonstrated that replicon RNAs produced in cells from the transfected plasmid DNAs DNALeuMpt and DNAProMpt were replication-competent. Northern blot analysis with KUN-specific cDNA probe of total RNA isolated from cells electroporated with in vitro synthesized RNAs RNALeuMpt and RNAProMpt (FIG. 2B) confirmed the IF results (FIG. 2A) and together they demonstrated efficient replication and accumulation of KUN replicon RNA in transfected cells. Note that twice more total cellular RNA was loaded on the gel for RNA-transfected samples than for DNA-transfected samples (FIG. 2B) and also that ethidium bromide staining of the gel showed similar concentration of ribosomal RNAs in corresponding (RNA-transfected or DNA-transfected) samples (data not shown).

To examine whether the Mpt sequence was retained during replication of replicon RNAs in cells, the membrane containing the same RNA samples (left panel in FIG. 2B) was re-probed with radiolabelled cDNA probe specific for Mpt sequence. The results (right panel in FIG. 2B) were identical to those obtained using KUN-specific probe (left panel in FIG. 2B) and clearly demonstrated that Mpt sequence was indeed retained during amplification of KUN replicon RNAs in transfected cells.

The purpose of generating Leu- and Pro-containing constructs was to evaluate a possible effect of this mutation at the amino acid position 250 in the KUN NS1 gene on the replication and accumulation of KUN replicon RNA and thus on the levels of expression of encoded HGs both in vitro and in vivo. It was previously shown that a Pro (wild type) to Leu mutation at the amino acid 250 in the KUN NS1 gene resulted in the loss of dimerization of the NS1 protein. This caused a delay in virus replication in Vero cells and attenuation of virus replication in mice (16). Infection of Vero cells with equal amounts of Leu- and Pro-containing viruses produced 100-fold less of the Leu-containing virus in the first 24 h, but similar yields of Leu- and Pro-containing viruses were obtained by 48 h after infection. In in vivo experiments, 100-fold more Leu-containing virus was required to generate similar clinical symptoms of the disease in mice (16). In the current study with KUN-Mpt replicon constructs, the amounts of KUN RNA accumulated in BHK21 cells transfected with DNA-based Leu- and Pro-containing replicon constructs were similar (compare DNALeuMpt and DNAProMpt lanes in FIG. 2B), while transfections with in vitro transcribed RNAs showed slightly higher levels of RNA accumulation in cells transfected with Leu-containing RNA than with Pro-containing RNA (compare RNALeuMpt and RNAProMpt in FIG. 2B). As mentioned above, the amounts of ribosomal RNAs were similar in corresponding samples as judged by ethidium bromide staining. Transfection of RNALeuMpt RNA also resulted in slightly higher proportion of NS3-positive cells in IF analysis (FIG. 2A). Noticeably, a significantly higher number of rounded (dead) NS3-positive cells were seen after transfection with RNAProMpt RNA than after transfection with RNALeuMpt RNA. (FIG. 2A), suggesting higher cytopathicity of the former RNA. Similar difference in cytotopathicity between Pro- and Leu-containing variants was also observed following transfections of corresponding KUN replicon vector RNAs RNAPro and RNALeu (data not shown).

Induction of CTL Responses Following Immunization with DNA-, RNA-, and VLP-Based KUN Replicons Expressing a Polytope Immungen.

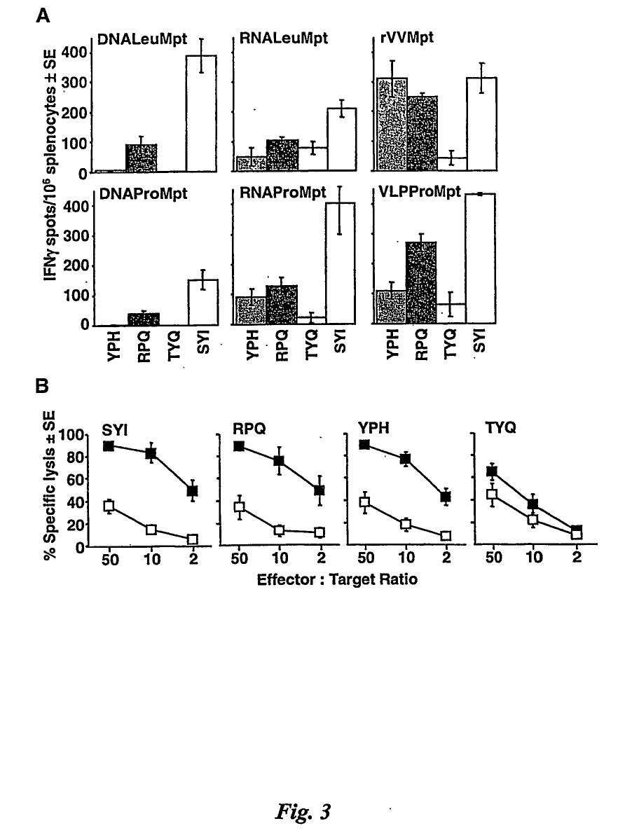

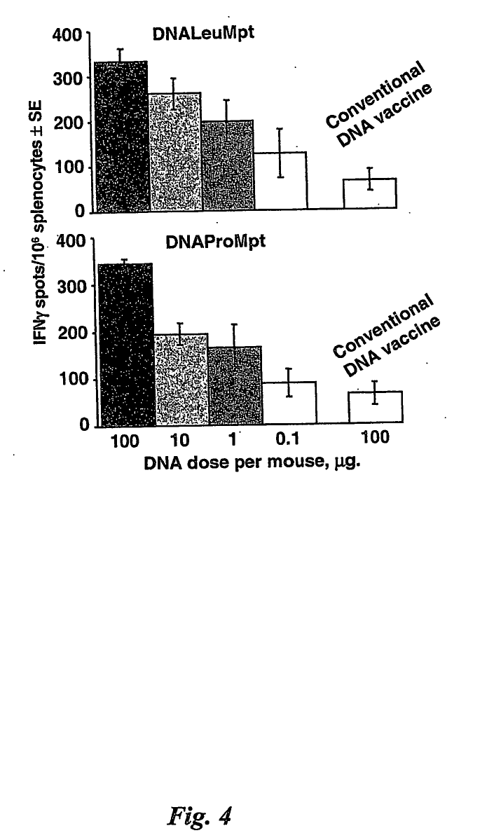

The murine polytope immunogen (Mpt) contains four conjoined CTL epitopes restricted by H-2d; these include YPHFMPTNL (a H-2Ld restricted epitope from murine cytomegalovirus pp89), RPQASGVYM (H-2Ld restricted epitope from LCMV nucleoprotein), TYQRTRALV (H-2Kd epitope from influenza virus nucleoprotein) and SYIPSAEKI (H-2Kd epitope from P. Berghei circumsporozoite protein). Balb/c mice were immunized once with KUN replicon DNA, RNA and VLPs encoding the Mpt immunogen and after 2-3 weeks CTL responses to each of the four CTL peptide epitopes was measured using IFNγ ELISPOT (FIG. 3A). The ELISPOT responses generated by the DNA, RNA, or VLP KUN vaccines were comparable with those induced by a recombinant vaccina virus encoding the same polytope immunogen (rVVMpt) (FIG. 3A) and were significantly higher than those induced by a conventional DNA vaccine encoding the same immunogen (see FIG. 4) (31). The hierarchy of epitope responses, with the RPQ-specific responses dominating over the YPH responses and the SYI responses dominating the TYQ responses have been observed previously and are thought to reflect the lower MHC-binding affinity of the subdominant epitopes (31). Although Pro-containing replicons appeared to be more cytopathic than Leu-containing replicons in transfected BHK21 cells (see previous section), no significant differences between these variants were observed in induction of CTL responses (FIG. 3A and see FIG. 4).

Mice immunized once with VLPProMpt were also assayed for the induction of CTL responses by ⁵¹Cr-release assay. Significant CTL activity specific for each epitope was observed (FIG. 3B). These responses were again comparable with those observed previously in mice immunized once with rVVMpt (51) and higher than those seen previously in mice immunized twice with a conventional DNA vaccine encoding the polytope immunogen (50).

Immunization of mice with ten-fold serial dilutions of the DNA-based KUN-polytope replicons DNALeuMpt and DNAProMpt illustrated that CTL responses were still detectable after a single immunization with only 0.1 μg of KUN replicon DNA (FIG. 4). The magnitude of CTL responses induced by 0.1 μg the KUN vaccine were comparable to those induced by immunization with 100 μg of conventional plasmid DNA-polytope vaccine (FIG. 4).

The data presented in this section illustrate that KUN replicon-based vaccines, delivered by any of the three different modalities (DNA, RNA, and VLPs), can efficiently induce CTL, generating responses similar in magnitude to recombinant vaccinia virus-based vectors and significantly higher than conventional DNA vaccines.

Immunization With Naked RNA or VLPs Encoding Polytope Protects Mice From Viral and Tumour Challenges.

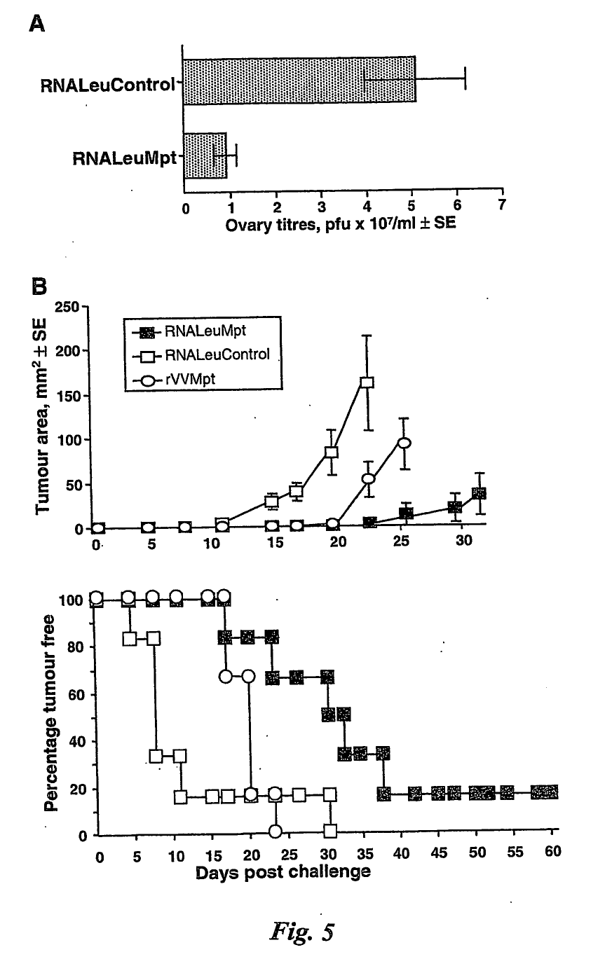

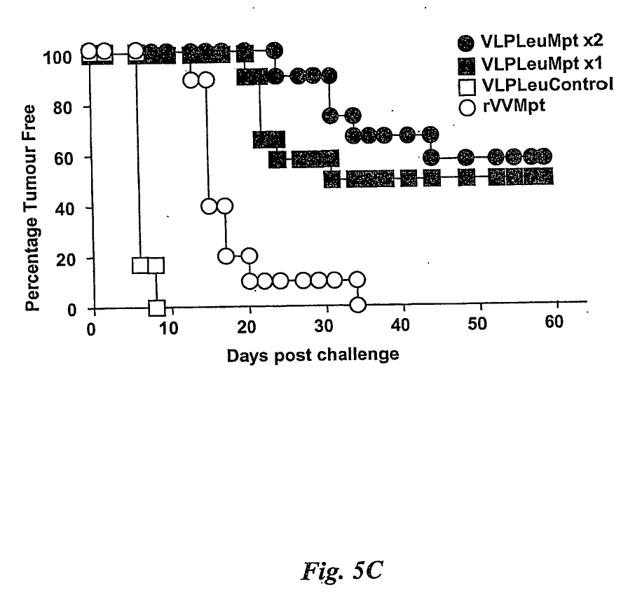

To determine whether the CTL responses induced by immunization with KUN replicon vectors can protect against viral challenge, mice were vaccinated once with RNALeuMpt RNA or RNALeuControl RNA (prepared from the RNALeu vector DNA) and were then challenged with rVVMpt. The rVVMpt challenge assay measured protection mediated through all four H-2d restricted epitopes presented by the KUN vaccine. Following a single vaccination with RNALeuMpt RNA a significant (≈70%) reduction in ovary rW titres was observed (FIG. 5A; p=0.03). Comparable protection was obtained using a conventional DNA-polytope vaccine but only after two immunizations (50).

The Mpt sequence also encoded the ovalbumin-derived CTL epitope, SIINFEKL (50, 51), permitting examination of tumour protection following KUN vaccination using challenge with B16 tumour cells expressing ovalbumin (B16-OVA). Mice were immunized twice with RNALeuMpt and RNALeuControl or once with rVVMpt and then challenged with B16-OVA. Significantly slower rate of tumour growth was observed in RNALeuMpt-immunized mice compared with RNALeuControl-immunized animals, (p<0.001) (FIG. 5B, upper graph). Furthermore, the average rate of tumour growth in RNALeuMpt-immunized mice was also slower than that following immunization with rVVMpt (p=0.001). The same experiment is also presented as Kaplan-Meier curves (FIG. 5B, lower graph), which show the percentage of mice surviving at the indicated time points. Again, RNALeuMpt-immunized mice performed significantly better than RNALeuControl-immunized animals (p=0.015) and than rVVMpt-immunized animals (p=0.016).

Referring to FIG. 5C, the data show that immunization with VLPLeuMpt greatly enhanced survival of immunized mice from B 16-OVA tumour challenge compared to immunization with naked RNA. It is also noted that mice receiving two VLP immunizations (VLPLeuMptx2) had measurably improved survival rates compared to mice receiving one VLP immunization (VLPLeuMptx1).

Generation and Evaluation of a Modified Viral Packaging System

Three different approaches were employed to modify a previously developed Kunjin replicon RNA packaging system described in International Publication WO 99/28487 in order to improve packaging efficiency and to simplify the packaging protocol.

(i) A first approach involved modification of the SFV-derived packaging construct SFV-MEC105 to decrease its cytopathicity by introducing mutation of amino acid 713 from leucine to proline (SFVMEC/L713P; FIG. 7). This resulted in prolonged expression of Kunjin structural genes without adverse effects of SFV RNA replication and allowed simplification of the packaging protocol by simultaneously transfecting both Kunjin and SFVMEC/L713P RNAs. The results are presented in Tables 1 and 2. Previous attempts to use simultaneous transfection of Kunjin and SFV-MEC1O5 RNAs resulted in complete inhibition of Kunjin RNA replication.

(ii) A second approach involved generation of a stable cell line continuously producing Kunjin structural proteins from the modified noncytopathic SFV replicon RNA. To achieve this, the present inventors inserted an IRES-Neo cassette into the SFVMEC/L713P construct (SFVMEC/L713P/Neo; FIG. 8) and established a stably expressing cell clone by selection with antibiotic G418. Transfection of this cell line with Kunjin replicon RNAs allowed its replication and production of relatively high titres of Kunjin VLPs (Tables 3 and 4).

(iii) A third approach was to generate a cell line stably expressing noncytopathic SFV replicon RNA pSFV3L713PLacZNeo (FIG. 9) to use it for amplification of transfected SFV helper RNAs pSFVHelperprMC (FIG. 10) and pSFVHelperCprME (FIG. 11) to express Kunjin structural proteins. When either of these helper RNAs are transfected together with Kunjin replicon RNA into pSFVL713PIacZNeo cell line, both SFV helper RNA and KUN replicon RNA are amplified and this results in production of secreted Kunjin VLPs, albeit with relatively low titres (Table 5).

Comparative Efficiencies of Kunjin VLP Production Between Cytopathic and Noncytopathic SFV-Derived Packaging Constructs.

Initially, BHK21 cells were electroporated with Kunjin replicon RNA (containing heterologous genes) transcribed from constructs such as RNALeuMpt. Then at 32 h, these Kunjin RNA-transfected cells were electroporated with either cytopathic (cyto) or noncytopathic (noncyto) Semliki Forest virus RNAs transcribed from pSFVMEC105 or pSFVMECL713P, respectively. The tissue culture fluid containing secreted VLPs was then harvested at different times after the second transfection as indicated in Table 1.

Alternatively, the Kunjin replicon RNA and the noncytopathic SFV RNA were transfected into BHK21 cells simultaneously and the tissue culture fluid was harvested at various time intervals as indicated in Table 2.

It should also be noted that different ratios of Kunjin RNA to SFV RNA as indicated in Tables 1 and 2 was used to aid optimisation of the level of VLP expression.

Kunjin VLP Production in Stable Cell Line Expressing Kunjin Structural Proteins From Modified Noncytopathic SFV-Derived Packaging Construct After Transfection With Kunjin Replicon RNAs

Stable cell line, SFVMECA12, expressed Kunjin structural proteins from noncytopathic SFV replicon for production of VLPs. This cell line expressed the noncytopathic SFV replicon encoding Kunjin structural proteins. To establish this cell line, BHK21 cells were transfected with SFVMECL713PNeo RNA (FIG. 8), and one clone, A12, was selected by incubation in the media with 1 mg/ml of G418. This cell clone was then evaluated by electroporation with Kunjin replicon RNA encoding heterologous genes such as HIV GAG or murine polytope (Mpt). Following electroporation with Kunjin replicon RNA, tissue culture fluid was harvested at different time points and the titre of VLPs calculated via an infection assay and immunofluorescence (Table 3).

Further experiments evaluated the effect of varying temperatures of incubation on VLP production. That is, following electroporation of the SFVMECA12 cells with Kunjin replicon RNAs, cells were incubated at 37° C., 33° C. or 37° C./33° C. (37° C. overnight then 33° C. until 50 h post-transfection (Table 4).

Production of Kunjin VLPs in Stable Cell Line Expressing Noncytopathic SFV Replicon After Co-Transfections With SFVHelper RNAs Expressing Kunjin Structural Proteins and With Kunjin Replicon RNAs.

A stable cell line expressing the noncytopathic SFV replicon and Lac Z gene was established using the construct, pSFV3L713PlacZNeo. This cell line expressed the noncytopathic SFV replicon with L713P mutation, encoded Neo and LacZ gene. The level and uniformity of expression within these cells was monitored by LacZ expression. Three different clones were chosen for analysis of further VLP production, i.e., clone #C5, C6 and C11. Each cell clone was electroporated simultaneously with Kunjin replicon RNA and one of the two SFV-Kunjin Helper RNAs. The Kunjin replicon RNA encoded heterologous genes such as HIV GAG or Murine polytope (Mpt). The SFV-Kunjin Helper RNAs encoded either Kunjin CprME gene cassette under control of one 26S promoter (pSFVHelperCprME; FIG. 10) or Kunjin prME and C genes under the control of two separate 26S promoters (pSFVHelperprMEC; FIG. 11), cloned in the pSFVHelper2 vector. Tissue culture fluid was then harvested at 28 h and 45 h post-transfection to examine the level of particle production (Table 5).

### EXAMPLE 2

### Materials and Methods

Plasmids. The RNA-based and DNA-based KUN replicon vectors (C20UbHDVrep and pKUNrep1, respectively), containing mouse ubiquitin gene upstream and FMDV2A autoprotese sequence downstream of the cloning site, were used for construction of plasmids containing the HIV-l gag gene. Essentially, the complete HIV-1 gag gene was amplified by PCR from the plasmid, pBRDH2-neo, with primers gagBssHH-F (5′-ACCATGGGCGCGAGCATCGGTATTA-3′) and gagBssHI-R (5′-CTAAAGCGCGCCTTGTGACGAGGGGTC-3′). The PCR product was then digested with BssHII and inserted into the AscI site of the two KUN vectors to produce the plasmids, KUNRNAgag and KUNDNAgag, respectively.

Preparation of KUNgag VLPs. VLPs Were prepared essentially as described previously except that 3×10⁶ BHK21 cells were electroporated with ˜30 μg of in vitro-transcribed KUNgag RNA. At 32 h post-electroporation, the cells were trypsinised and subjected to a second electroporation using in-vitro transcribed RNA from a less cytopathic form of the construct SFVMEC105, SFVL713PMEC containing Leucine 713 to Proline substitution in the nsP2 gene. Following the second electroporation, cells were incubated at 37° C. for 18 h, then the medium was replaced with 6 ml of DMEM-5% FCS and the cells were incubated at 33° C. for a further 30 h. The titre of infectious VLPs was determined by infection of Vero cells with 10-fold serial dilutions of the VLPs and counting the number of NS3-positive cells by IF analysis at 30 to 40 h post-infection.

Immunization of mice. Female BALB/c (H-2^{d}) mice (6 to 8 weeks) were supplied by the Animal Resources Centre (Perth, Western Australia). Mice were immunized with one of the following (i) gag VLPs in DMEM-5% FCS which were injected intraperitoneally (ip.) at ˜1×10⁶ infectious units per mouse, and (ii) a recombinant vaccinia virus encoding HIV-1 gag (10⁷ pfu in 200 μl of RPMI 1640, i.p.)

Indirect ELISA. 96 well plates were coated with 1 μg of purified recombinant p55 antigen overnight at 4° C. in antigen coating buffer (15 mM Na₂CO₃, 35 mM NaHCO₃, pH 9.6). The wells were then blocked by incubation with 50 μl of blocking buffer (0.25% Gelatin/0.1% Tween-20 in PBS) for 1 h at 37° C. and washed 3 times with wash buffer (0.05% Tween-20 in PBS). Sera samples from immunized mice, diluted in blocking buffer, were incubated for 1-2 h at room temperature then washed 3 times. The secondary antibody, horseradish peroxidase (HRP)-conjugated goat anti-mouse IgG diluted 1:2000 in blocking buffer was incubated for 30 min at room temperature. After 3 washes, bound conjugate was developed by incubation with 50 μl of K-Blue TMB substrate (Graphic Scientific, Australia) 10 min at RT in the dark or until the colour developed. The reaction was then stopped by addition of 50 μl of 2 M H₂SO₄ and the OD₄₅₀ absorbance readings were determined using an ELISA plate reader.

Kunjin VLP neutralisation assay. 200 μl of KUN VLPs containing encapsidated KUN vector replicon RNA (5×10⁵ IU) were incubated for 1 h at 37° C. with 20 μl of 3 different serum samples from KUNgag VLP-immunized mice which showed the highest titres of gag antibodies by ELISA. VLPs were also incubated under the same conditions without antibody and with a KUN anti-E monoclonal antibody (Mab) as positive and negative controls for the assay. After incubation, the titre of each sample was determined by infection of Vero cells with dilutions of each sample. IF analysis was then performed using KUN anti-NS3 antibody and the positive foci were counted and the titre calculated.

Vaccinia virus protection assay. Groups (n=5-6) of 6- to 8-week old female BALB/c mice were immunized (i) once and twice (with a 4 week interval) with ˜10⁶ IU per mouse of KUNgag VLPs, and after 3 weeks challenged i.p. with 10⁷ PFU per mouse of recombinant vaccinia virus encoding HIV-1 gag (rVVgag, (14)). At day 4 post-infection, both ovaries were removed, washed and homogenised in 1 ml of PBS with a motorised grinder. The titre of vaccinia virus in ovary suspensions were determined by plaque assay on confluent CV1 cells. The significance of the differences between the virus titres in the experimental and control groups was calculated using the Wilcoxon rank-sum test.

Measurement of CD4+T cell responses. Female Balb/c (H-2d) mice (6-8 weeks old) were supplied by Animal Resource Centre (Perth, Wash.). Mice (n=4) were immunized intraperitoneally (ip) with KUNgag VLPs once (KUNgagVLP) or left unimmunized (naive). Two weeks after immunization, mice were culled and splenocytes (gag) or media only (Media) and over the last 18 hours, pulsed with ³H thymidine. The cells were harvested onto glass fibre mates and β-emissions were measured. Gag-pulsed naive splenocytes were prepared a day before the assay; splenocytes from naive mice were treated with RBC lysis buffer and pulsed overnight with HIV-1 gag protein in vitro at 2 μg/ml.

### Results

Induction of Antibody Responses to HIV Gag Protein Following Immunization With KUN Replicons Expressing HIV-1 gag.

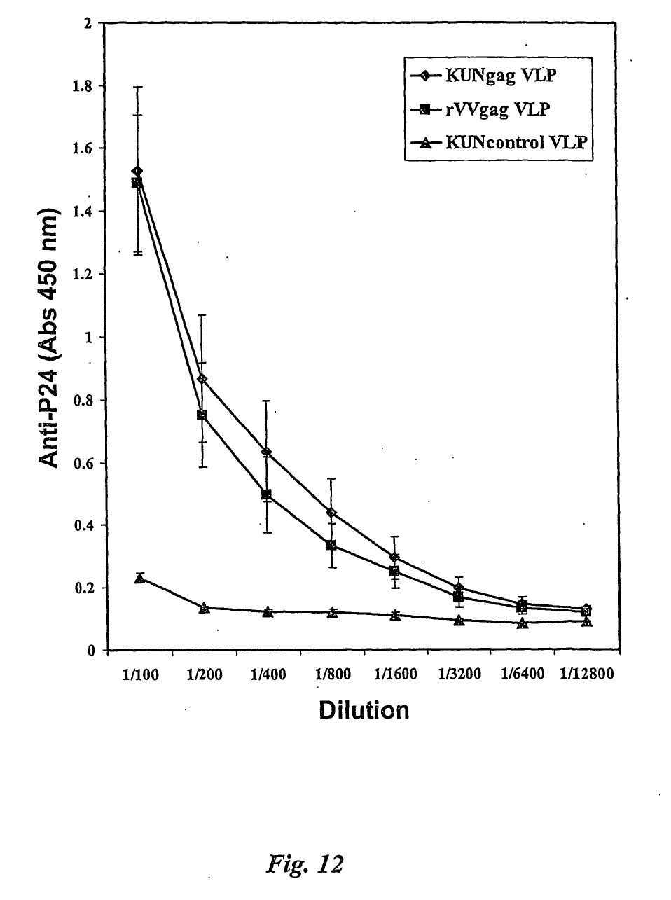

Sera from mice immunized twice with KUNgag VLPs (106 IU per mouse) or once with recombinant vaccinia virus encoding HIV-1 gag (rWgag, 10⁷ PFU per mouse), were examined for the presence of antibodies to gag. Six of 8 mice immunized with gag VLPs and 3 of 4 mice immunized with rWgag responded to immunizations and significant titres (>1:12800) of anti-gag antibodies were detected in these mice (FIG. 12). The antibody responses were similar in KUNgag VLPs and rWgag-immunized mice.

Absence of Netralizing Antibody Responses to KUN VLP Proteins After Immunization With KUN Replicon VLPs.

To determine if there was any significant neutralising antibody response to KUN envelope protein displayed on the surface of KUN VLPs after immunization with KUNgag VLPs, we performed an infectivity neutralisation assay by incubation of sera from KUNgag VLP-immunized and non-immunized mice with KUN VLPs containing encapsidated KUN vector replicon RNA. The titre of KUN VLPs incubated without any antibodies was 2.8×10⁶ IU/ml. The average titre of KUN VLPs incubated with 3 different sera from non-immunized mice was 2.1×10⁶ IU/ml. No infectivity was observed when KUN VLPs were incubated with monoclonal antibodies to KlN E protein. The average titre of KUN VLPs incubated with 3 different sera from mice immunized twice with KUNgag VLPs was 6.3×10⁵ Iu/ml. A nonparametric, unpaired Mann-Whitney test showed that there was no significant difference in the VLP titres obtained in neutralisation assays with sera from KUNgag RNA-immunized mice as compared to those obtained with sera from non-immunized mice (p<0.1). Thus, no significant neutralising antibody response to KUN proteins present in VLPs was observed after two immunizations with KUNgag VLPs.

VLP Particle Immunization With KUN Replicons Encoding HIV-1 Gag Protects Mice From Experimental Viral Challenge.

To determine whether the CD8+T cell responses induced by immunization with KUN replicon vectors can protect against viral challenge, groups of 6 mice were immunized once or twice with KUNgag VLPs or not immunized and then challenged with rVVgag. Three out of 6 mice (50%) in the group immunized once and 4 out of 6 mice (67%) in the group immunized twice, were completely free of the challenged virus (Table 1) demonstrating complete protection. One mouse within the KUNgag VLP ×1 group had almost the same titre of challenge virus as the non-immunized mice (2×10⁷) indicating immunization failure. Thus, the average titre in the remaining two responding mice after the single and double immunizations with KUNgag VLPs were 6.7×10⁶ IU/ml and 1.3×10⁵ IU/ml, respectively, showing up to greater than a 6 log reduction of viral replication in mice immunized once or twice, from the 10⁷ PFU of challenge virus, rVVgag. The results presented in this section demonstrated that KUN replicon-based vaccines encoding the HIV-1 gag gene induced HIV-1 gag-specific protective CTL responses, with a single VLP immunization providing much more efficient protection than a double immunization with naked RNA.

Kunjin VLP Encoding EIIV-1 Gag Elicits Gag-Specific CD4+T Cell Responses.

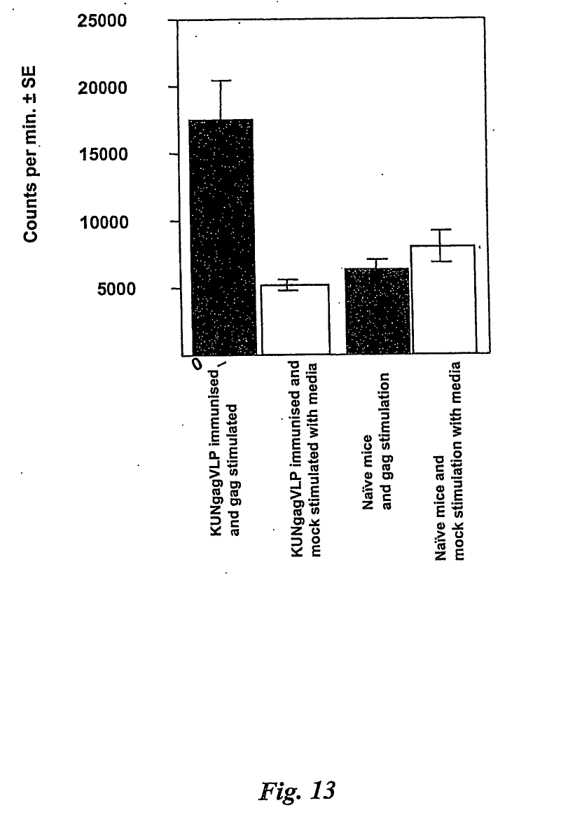

Experiments were performed to determine whether antibody production against HIV-1 gag was accompanied by T cell help provided by CD4+T cells. The data shown in FIG. 13 indicate that for VLP gaga-immunized mice, the stimulation index (SI) was 3.4, whereas for naive mice the SI was close to zero. These data demonstrate that VLPgag-induced specific, CD4 T cell responses in immunized mice.

### EXAMPLE 3

Induction of Long Term Protective CD8 T Cell Responses Methods

Preparation of VLPs. VLPs were prepared essentially as described previously except that 3×10⁶ BHK21 cells were electroporated with ˜30 μg of in vitro-transcribed KUNgag RNA. At 32 h post-electroporation, the cells were trypsinised, subjected to a second electroporation using in-vitro transcribed noncytopathic Semliki Forest virus replicon RNA encoding KUN structural proteins (SFV-L713PMEC105 derivative of SFV-MEC105; ref 28) and incubated for 48 h before harvesting secreted VLPs. The titre of infectious VLPs was determined by infection of Vero cells with 10-fold serial dilutions of the VLPs and counting the number of NS3-positive cells by IF analysis at 30 to 40 h post-infection. Immunization of mice. Female BALB/c (H-₂d) mice (6 to 8 weeks) were supplied by the Animal Resources Centre (Perth, Western Australia). Mice were immunized with one of the following (i) 100 μg of KUNgag DNA diluted in 100 μl PBS and injected intramuscularly (i.m.) into the quadriceps muscle of each hind leg (50 μl in each leg), (ii) 30 μg of in vitro transcribed KUNgag RNA, dissolved in 100 μl diethyl pyrocarbonate-treated PBS and injected im. (50 μl into each leg), (iii) KUNgag VLPs in Dulbecco modified Eagle medium (DMEM)-5% fetal calf serum (FCS) which were injected intraperitoneally (ip.) at ˜1×10⁶ infectious units per mouse, (iv) a KUN VLP encoding the murine polytope (KUNmpt VLP) which contain 4 H-2d restricted epitopes, YPHFMPTML (YPH), RPQASGVYM (RPQ), TYQRTRALV (TYQ) and SYIPSAEKI (SYI) (60) and was injected as for (iii),.and (v) recombinant vaccinia virus encoding HIV-1 gag (WR TK⁻) encoding HIV-1 gag (rWgag,) (2×10⁷pfu in 200 μl of PBS, ip.).

ELISPOT and chromium release assays. Epitope-specific IFNγ-secreting CD8 T cells were enumerated by an enzyme-linked immunospot (ELISPOT) assay using peptide epitopes (Mimotopes, Clayton, Australia) as described previously (34). The significance of differences between groups was determined using an unpaired Students t test. ⁵¹Chromium (⁵¹Cr) release assays were performed using splenocytes from mice sacrificed 2 to 3 weeks post immunization, and splenocytes were re-stimulated in vitro for 6 days with irradiated LPS blasts (responder:stimulator ratio 20:1) sensitized with the AMQMLKETI peptide (25 μg/ml for 1 h in 200 μl medium at 37° C. followed by 2 washes). The resulting effector populations were split, and equal numbers were used in duplicate against peptide-sensitized and unsensitized ⁵¹Cr-labelled P815 target cells at the indicated effector:target ratios.

Vaccinia virus protection assay. Groups (n=6) of 6-8-week old female BALB/c mice (Animal Resource Center, Perth, Australia) were immunized and control mice where challenged challenged i.p. with 10⁶ pfu per mouse of recombinant vaccinia virus (WR TK⁻) encoding HIV-1 gag (rVVgag) or the same dose of rVVmpt (ref. 31) At day 4 post infection, both ovaries were removed, washed and homogenized in I ml of PBS using aluminum mesh. The ovary vaccinia virus titers were then determined by plaque assay on confluent CV1 cells. The significance of the differences between the virus titers in the experimental and control groups was calculated using a non parametric unpaired t test.

### Results

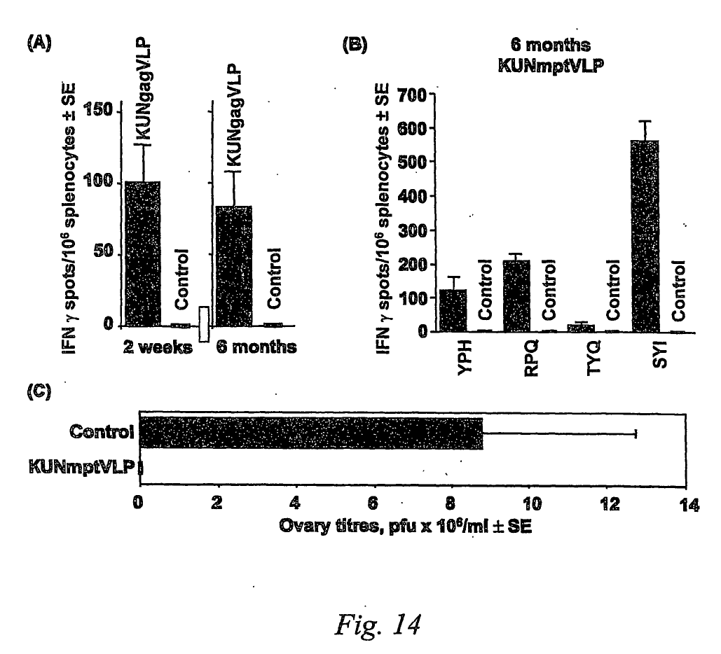

Mice were immunized with a single inoculation of KUNgag VLPs and the AMQ-specific responses were analyzed by ELISPOT after 2 weeks and 6 months. The responses at 6 months were not significantly lower than those seen at 2 weeks (p=0.75, FIG. 14A), illustrating the induction of long lasting CD8 T cell responses by the KUNgag VLP vaccine. A similar long-term maintenance of epitope-specific CD8 T cells capable of secreting IFNγ was observed following immunization with a KUN VLP encoding the murine polytope (KUNmptVLP), a vaccine encoding four H-2d restricted CD8 T cell epitopes. We have earlier described the responses to these four epitopes 2-3 weeks after immunization with KUNmptVLP and now show that after 6 months these responses have not significantly diminished (FIG. 14B), illustrating that long term maintenance of CD8 T cell responses is not restricted to AMQ-specific CD8 T cells. Furthermore, when mice, which had been immunized with KUNmptVLPs 10 months previously, were challenged with a recombinant vaccinia virus encoding the same four epitopes, substantial protection was still apparent (FIG. 14C). KUNmpt VLP immunized mice showed an average of about 120 fold reduction in ovary titers compared with control mice (p=0.015), with 83% of the KUNmptVLP immunized mice completely free of the challenged virus.

### EXAMPLE 4

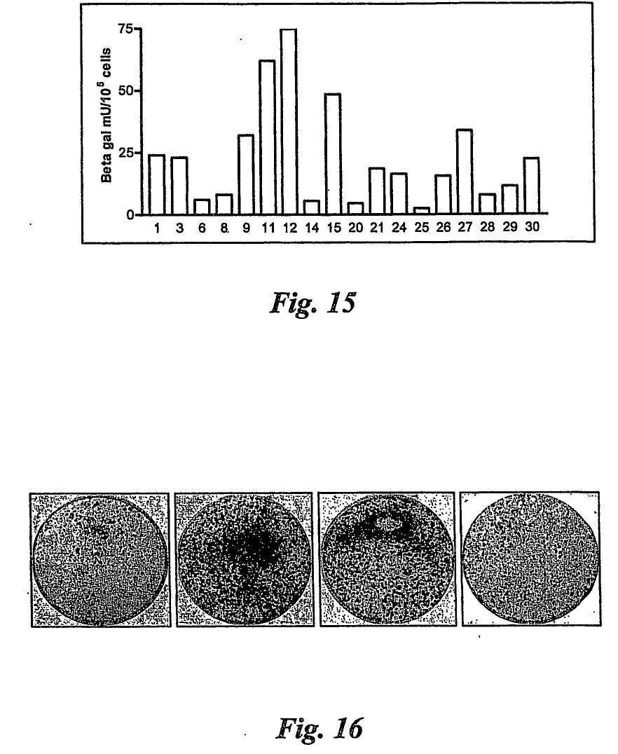

Selection of KUN replicon mutants adapted to rersistent replication in BK cells selection of puromycin resistant BHK cell clones arbouring persistently replicating KUN replicon RNA. BHK21 cells were electroporated with in vitro transcribed repPACβ-gal RNA which has identical to the wild type KUN cDNA sequence including Pro at position 250 in NS1 (ref. 16). Puromycin resistant cell clones were established by incubating in the medium containing 5 μg/ml puromycin for 7 days. Individual cell clones were picked and propagated in puromycin-containing medium for 4 passages. Cells were 100% β-gal positive as detected by X-gal staining and β-gal expression of different cell clones was determined by β-galactosidase assay kit (Promega). A wide range of P-gal expression levels was observed among different cell clones (FIG. 15). Two contrasting clones No. 11 and No. 20 showing different levels of expression were selected for sequencing analysis of KUN replicon RNA.

Determination of adaptive mutations in KUN replicon RNA from two selected cell clones. KUN replicon RNA from the puromycin resistant cell clones No.11 and No.20 was isolated using Absolutely RNA kit (Qiagen), amplified by RT-PCR and the entire KUN replicon sequence (except β-gal gene) was determined by sequencing analysis using Big Dye sequencing kit (Perkin Elmer). One adaptive mutation was found in KUN RNA isolated from clone 20 (Asn101 to Asp in NS2A), and two adaptive mutations were found in KUN RNA isolated from clone 11 (Ala30 to Pro in NS2A and Pro270 to Ser in NS5) (Table 7).

Adaptive mutations confer an advantage for establishment of persistent replication in BHK cells. The adaptive mutations identified in KUN replicon RNAs isolated from clone 11 and clone 20 were introduced individually into the original repPACβ-gal. construct by site directed PCR mutagenesis to generate repPACβ-gal/NS2A(A-P), repPACβ-gal/NS2A(N-D) and repPACβ-gal/NS5(P-S). BHK cells were electroporated with the wild type and mutated RNAs, and the effect of adaptive mutations was evaluated by the differences in the number of puromycin resistant cell colonies at 4 days after addition of puromycin (FIG. 16). As evident from FIG. 16, transfection of RNA containing mutations resulted in selection of higher number of puromycin resistant cell colonies than the wild type replicon, with Ala30 to Pro mutation in NS2A conferring the most efficient adaptation to persistent replication in BHK cells.

It has also been found by the present inventors that the same mutations confer an advantage for establishment of persistent replication in HEK293 cells

### General Discussion

The present inventors have demonstrated the ability of KUN replicon vectors to induce CTL to an encoded model immunogen after a single immunization. All three delivery modalities, i.e. naked RNA, plasmid DNA, and VLPs, were efficient in induction of CD8+CTL responses. Immunization with naked replicon RNA and VLPs also protected mice from virus and tumour challenges. These results demonstrate the potential of KUN replicon based vaccine vectors for the induction of protective T cell responses including CD8+CTLs and CD4+T cells. The magnitude of CTL responses obtained using KUN replicon vectors were comparable to or better than those obtained with recombinant vaccinia virus, a vaccine vector modality, widely regarded as an effective vector for CTL induction (44). A number of poxviral vaccine vectors are currently being tested for their ability to induce protective CTL in primates and humans; these include modified vaccinia Ankara (48), fowlpox virus (43), and ALVAC (15).

Alphavirus replicons have been extensively used for the induction of immune responses in animal models and replicons derived from VEE virus have recently been approved for pre-clinical trials in humans (http://www.alphavax.com/p_inhouse.html). The results herein show that KUN replicons can be delivered by three different modalities: as naked RNA, as plasmid DNA, and as VLPs. Immunization with only 0.1 μg of KUN DNA-based replicon was sufficient to elicit CTL responses. A similar level of CTL induction was achieved only after immunization with a 1000-fold higher dose (100 μg) of a conventional plasmid DNA vaccine (FIG. 4).

A particular aspect of the present invention relates to RNA immunization. Naked RNA immunization avoids issues relating to DNA integration and may offer additional advantages over immunization with VLPs. In contrast to naked RNA, immunization with VLPs is likely to lead to the induction of neutralizing antibody responses to the structural proteins of VLPs, a phenomenon which may limit the number of times the VLPs can be used in one individual. RNA-based vaccines are also easier to manufacture than VLPs. Recent reports also suggest that RNA can be encapsulated into microparticles and delivered into animals by bombardment with a gene gun (37, 54). Encapsulated RNA was also shown to be stable for 8-12 months at 4° C.

The present inventors have shown that RNA immunization with KUN replicon vaccines resulted in the induction of CTL responses specific for an encoded immunogen with efficiency comparable to that obtained after immunizations with DNA-based or VLP-based KUN replicons (FIG. 3A). RNA vaccination using KUN or SIN replicons also induced significant anti-tumour protection in different tumour models (FIG. 5B) (58).

One particular feature of Flaviviruses may prove to be beneficial for the use of their replicons as a safe vaccine vectors. Flaviviruses in general, have shown no evidence of recombination between different viruses or virus strains in nature. Our previous numerous complementation experiments with defective full-length KUN RNAs and replicon RNA as a helper also failed to show any evidence of recombination between these two (full-length and replicon) RNAs co-replicating in the same cell despite the presence of extended regions of perfect homology in both RNAs (summarized in reference 29). No recombination leading to the recovery of recombinant viruses were also detected in complementation experiments with yellow fever virus RNAs containing deletions in the NS1 gene when supplying the helper NS1 protein either from homologous (yellow fever) or from heterologous (dengue) viruses (34, 35). This feature of Flaviviruses is in contract to the well-documented recombination abilities of alphavirus RNAs (18, 42, 46), and may provide an extra level of safety for vaccinations of individuals, which may be infected with the same or other flaviviruses either just prior of some time after immunization. In addition, the heterologous nature of the KUN replicon packaging system employing a replicon RNA-based vector from a totally unrelated virus, SFV, for the expression of KUN structural proteins, as well as a specific design of the expression construct make KUN VLP preparations absolutely safe and free of any infectious recombinant viral material (28, 52).

Perhaps one of the most distinctive features of the KUN replicons is that their replication does not cause an overt cytopathic effect (CPE) or apoptosis (26, 52, 53). CPE or apoptosis of infected cells containing vaccine antigens was reported to be an efficient method for cross priming, resulting in the delivery of vaccine antigens to dendritic cells (DCs) and effective CTL induction (2, 58). However, despite exhibiting a very low/undetectable level of cytopathicity in vitro (Leu-containing variants; FIG. 2A), KUN replicons efficiently induced CTL responses in mice (FIGS. 3 and 4). Furthermore, no significant differences were observed in the CTL induction by the Leu- and Pro-containing variants of the KUN replicon vectors (FIGS. 3 and 4), which had showed different degrees of cytopathicity in transfected BHK21 cells (FIG. 2A). CTL induction by KUN replicons may thus be independent of apoptosis-mediated cross priming (5) and/or may not require cross priming. It has been shown that flaviviruses can directly infect, replicate and activate DCs (19, 21, 22, 32, 57), suggesting that KUN replicons may also be able to replicate and produce vaccine antigens in DCs. Direct infection of DCs by vaccine vectors may represent a more efficient strategy for CTL induction than cross priming (49). Lymphotropic VEE replicon particles as well as DC-adapted SIN replicon particles were recently shown to be able to infect DCs and express an encoded vaccine antigen (14, 36). However, the cytopathic nature of alphavirus replicon RNAs may lead to apoptosis of antigen-presenting DCs and a reduction in CTL activation or induction. In contrast, KUN replicons with limited level of cytopathicity may allow prolonged antigen expression in DCs without causing apoptosis; a feature, which may contribute to prolonged stimulation of CTL responses. Induction of tolerance is unlikely, due to the ever-present double-stranded RNA, which is believed to induce danger signals and activate DCs (13).

Perhaps the most impressive outcome of KUN VLP immunization is the maintenance for 6-10 months of CD8+T cell responses that are capable of immediate IFNγ secretion and of mediating protection within the four days of the rVV challenge assay (FIG. 14). Such long-term maintenance of effector CD8 T cells capable of immediate protective activities may emerge as an important feature of effective vaccination against HIV, since the post-challenge generation of new effectors from a vaccine-induced memory CD8 T cell pool may simply be too slow to deal effectively with the explosive retroviral infection.

In summary, KUN replicons have been shown to be effective vaccine vectors for the induction of long term, protective CD8+T cells and may represent an attractive new modality for cancer and H[V vaccines.

Throughout this specification, the aim has been to describe the preferred embodiments of the invention without limiting the invention to any one embodiment or specific collection of features. Various changes and modifications may be made to the embodiments described and illustrated herein without departing from the broad spirit and scope of the invention.

All computer programs, algorithms, patent and scientific literature referred to in this specification are incorporated herein by reference in their entirety.

TABLE 1

| | Titres (infectious units/ml) at different times | | | |
|---|---|---|---|---|
| | after 2^{nd} transfection | | | |
| KUN/SFV ratio | 23 h | 37 h | 45 h | 61 h |
| KUN/SFVMEC105 | 3.8 × 10⁴ | 7.8 × 10⁴ | 1.8 × 10⁵ | 1.6 × 10⁵ |
| 1:1 | | | | |
| KUN/SFVL713MEC | 8 × 10³ | 1.5 × 10⁴ | 2.3 × 10⁴ | 2.6 × 10⁴ |
| 1:1 | | | | |
| KUN/SFVL713MEC | 3 × 10³ | 2.8 × 10⁴ | 5.1 × 10⁴ | 4.8 × 10⁴ |
| 2:2 | | | | |
| KUN/SFVL713MEC | 5.8 × 10⁴ | 1.1 × 10⁴ | 1.4 × 10⁴ | nd |
| 1:2 | | | | |

TABLE 2

| | Titre (infectious units/ml) at different time | | | |
|---|---|---|---|---|
| | after transfection | | | |
| KUN/SFV ratio | 24 h | 42 h | 51 h | 66 h |
| KUN/SFVL713MEC | 1 × 10² | 2.3 × 10³ | 3.5 × 10³ | 9.4 × 10³ |
| 1:1 | | | | |
| KUN/SFVL713MEC | 4 × 10² | 4.4 × 10³ | 7.1 × 10³ | 9 × 10³ |
| 2:2 | | | | |
| KUN/SFVL713MEC | 0 | 1.9 × 10³ | 5.1 × 10³ | 9.2 × 10³ |
| 1:2 | | | | |

TABLE 3

| | VLP titres (infectious units/ml) at different times after | | | |
|---|---|---|---|---|
| | transfection | | | |
| | 39 h | 48 h | 63 h | 72 h |
| Kun-GAG VLPs | 2 × 10⁵ | 2.6 × 10⁵ | 1.9 × 10⁵ | 2 × 10⁵ |
| Kun-Mpt VLPs | 7 × 10⁵ | 2.7 × 10⁵ | 2.3 × 10⁵ | — |

TABLE 4

| | |
|---|---|
| SFVMECA12 cells + Kun | Titre (infectious units/ml) |

| GAG RNA | Experiment 1 | Experiment 2 |
|---|---|---|
| 37° C. 50 h | 6 × 10⁵ | 1.4 × 10⁵ |
| 33° C. 50 h | 1.3 × 10⁶ | 5 × 10⁴ |
| 37° C. 18 h/33° C. 32 h | — | 4 × 10⁵ |

TABLE 5

| Cell | | Titre (Infectious units/ml) | |
|---|---|---|---|
| Clone # | RNAs transfected | 28 h | 45 h |
| C5 | SFVHelperCprME + KUN- | 1.3 × 10³ | 3 × 10⁴ |
| | GAG | | |
| C6 | SFVHelperCprME + KUN- | 2.9 × 10⁴ | 5.7 × 10⁴ |
| | GAG | | |
| C11 | SFVHelperCprME + KUN- | 2 × 10⁴ | 1.5 × 10⁵ |
| | GAG | | |

TABLE 6

| Groups | rVVGag titre | Fold reduction |
|---|---|---|
| Control | 2.8 × 10⁷ | |
| (n = 5) | | |
| KUNgag | 3 mice (50%) - <10 | >2500000 |
| VLP × 1 | 1 mouse - 2 × 10³ | 14000 |
| (n = 6) | 1 mouse - 3.5 × 10⁴ | 800 |
| | 1 mouse - 2 × 10⁷ | 1.4 |
| KUNgag | 4 mice (67%) - <10 | >2500000 |
| VLP × 2 | 1 mouse - 1.5 × 10⁴ | 1867 |
| (n = 6) | 1 mouse - 2.5 × 10⁵ | 112 |

TABLE 7

| protein | | | | |
|---|---|---|---|---|
| position | amino acid | wild type | Clone 11 | Clone 20 |
| NS2A | 30 | Ala (A) | Pro (P) | — |
| NS2A | 101 | Asn (N) | — | Asp (D) |
| NS5 | 270 | Pro (P) | Ser (S) | — |

### REFERENCES

1. Agapov, E. V., I. Frolov, B. D. Lindenbach, B. M. Pragai, S. Schlesinger, and C. M. Rice. 1998. Noncytopathic Sindbis virus RNA vectors for heterologous gene expression. Proc Natl Acad Sci U S A. 95:12989-12994.
2. Albert, M. L., B. Sauter, and N. Bhardwaj. 1998. Dendritic cells acquire antigen from apoptotic cells and induce class I-restricted CTLs. Nature. 392:86-89.
3. Bellone, M., D. Cantarella, P. Castiglioni, M. C. Crosti, A. Ronchetti, M. Moro, M. P. Garancini, G. Casorati, and P. Dellabona. 2000. Relevance of the tumor antigen in the validation of three vaccination strategies for melanoma. J Immunol. 165:2651-2656.
4. Berglund, P., C. Smerdou, M. N. Fleeton, I. Tubulekas, and P. Liljestrom. 1998. Enhancing immune responses using suicidal DNA vaccines. Nat Biotechnol. 16:562-565.
5. Binder, R. J., D. K. Han, and P. K. Srivastava. 2000. CD91: a receptor for heat shock protein gp96. Nat Immunol. 1:151-155.
6. Bredenbeek, P. J., I. Frolov, C. M. Rice, and S. Schlesinger. 1993. Sindbis virus expression vectors: packaging of RNA replicons by using defective helper RNAs. J Virol. 67:6439-6446.
7. Conover, W. J. 1980. Practical noparametric satistics. Wiley, New York.
8. Davis, N. L., I. J. Caley, K. W. Brown, M. R. Betts, D. M. Iribeck, K. M. McGrath, M. J. Connell, D. C. Montefiori, J. A. Frelinger, R. Swanstrom, P. R. Johnson, and R. E. Johnston. 2000. Vaccination of macaques against pathogenic simian immunodeficiency virus with Venezuelan equine encephalitis virus replicon particles. J Virol. 74:371-378.
9. Elliott, S. L., S. Pye, T. Le, L. Mateo, J. Cox, L. Macdonald, A. A. Scalzo, C. A. Forbes, and A. Suhrbier. 1999. Peptide based cytotoxic T-cell vaccines; delivery of multiple epitopes, help, memory and problems. Vaccine. 17:2009-2019.
10. Fleeton, M. N., P. Liljestrom, B. J. Sheahan, and G. J. Atkins. 2000. Recombinant Semliki Forest virus particles expressing louping ill virus antigens induce a better protective response than plasmid-based DNA vaccines or an inactivated whole particle vaccine. J Gen Virol. 81:749-758.
11. Fleeton, M. N., M. Chen, P. Berglund, G. Rhodes, S. E. Parker, M. Murphy, G. J. Atkins, and P. Liljestrom. 2001. Self-replicative RNA vaccines elicit protection against influenza A virus, respiratory syncytial virus, and a tickbome encephalitis virus. J Infect Dis. 183:1395-1398.
12. Frolov, I., E. Agapov, T. A. Hoffman, Jr., B. M. Pragai, M. Lippa, S. Schlesinger, and C. M. Rice. 1999. Selection of RNA replicons capable of persistent noncytopathic replication in mammalian cells. J Virol. 73:3854-3865.
13. Gallucci, S., M. Lolkema, and P. Matzinger. 1999. Natural adjuvants: endogenous activators of dendritic cells. Nat Med. 5:1249-1255.
14. Gardner, J. P., I. Frolov, S. Perri, Y. Ji, M. L. MacKichan, J. zur Megede, M. Chen, B. A. Belli, D. A. Driver, S. Sherrill, C. E. Greer, G. R. Otten, S. W. Barnett, M. A. Liu, T. W. Dubensky, and J. M. Polo. 2000. Infection of human dendritic cells by a sindbis virus replicon vector is determined by a single amino acid substitution in the E2 glycoprotein. J Virol. 74:11849-11857.
15. Gorse, G. J., G. B. Patel, and R. B. Belshe. 2001. HIV type 1 vaccine-induced T cell memory and cytotoxic T lymphocyte responses in HIV type 1-uninfected volunteers. AIDS Res Hum Retroviruses. 17:1175-1189.
16. Hall, R. A., A. A. Khromykh, J. M. Mackenzie, J. H. Scherret, T. I. Khromykh, and J. S. Mackenzie. 1999. Loss of dimerisation of the nonstructural protein NS1 of Kunjin virus delays viral replication and reduces virulence in mice, but still allows secretion of NS1. Virology. 264:66-75.
17. Hariharan, M. J., D. A. Driver, K. Townsend, D. Brumm, J. M. Polo, B. A. Belli, D. J. Catton, D. Hsu, D. Mittelstaedt, J. E. McCormack, L. Karavodin, T. W. Dubensky, Jr., S. M. Chang, and T. A. Banks. 1998. DNA immunization against herpes simplex virus: enhanced efficacy using a Sindbis virus-based vector. 3 Virol. 72:950-958.
18. Hill, K. R., M. Hajou, J. Y. Hu, and R. Raju. 1997. RNA-RNA recombination in Sindbis virus: roles of the 3′ conserved motif, poly(A) tail, and nonviral sequences of template RNAs in polymerase recognition and template switching. J Virol. 71:2693-2704.
19. Ho, L. J., J. J. Wang, M. F. Shaio, C. L. Kao, D. M. Chang, S. W. Han, and J. H. Lai. 2001. Infection of human dendritic cells by dengue virus causes cell maturation and cytokine production. J Immunol. 166:1499-1506.
20. Hosmer, D. E. a. L., S 1999. Applied survival analysis. Wiley, New York.
21. Johnston, L. J., G. M. Halliday, and N. J. King. 1996. Phenotypic changes in Langerhans' cells after infection with arboviruses: a role in the immune response to epidermally acquired viral infection? J Virol. 70:4761-4766.
22. Johnston, L. J., G. M. Halliday, and N. J. King. 2000. Langerhans cells migrate to local lymph nodes following cutaneous infection with an arbovirus. J Invest Dermatol. 114:560-568.
23. Kamrud, K. I., J. W. Hooper, F. Elgh, and C. S. Schmaljohn. 1999. Comparison of the protective efficacy of naked DNA, DNA-based Sindbis replicon, and packaged Sindbis replicon vectors expressing Hantavirus structural genes in hamsters. Virology. 263:209-219.
24. Kaul, R., S. L. Rowland-Jones, J. Kimani, T. Dong, H. B. Yang, P. Kiama, T. Rostron, E. Njagi, J. J. Bwayo, K. S. MacDonald, A. J. McMichael, and F. A. Plummer. 2001. Late seroconversion in HIV-resistant Nairobi prostitutes despite pre-existing HIV-specific CD8+responses. J Clin Invest. 107:341-349.
25. Khromykh, A. A. 2000. Replicon-based vectors of positive strand RNA viruses. Curr Opin Mol Ther. 2:555-569.
26. Khromykh, A. A., and E. G. Westaway. 1997. Subgenomic replicons of the flavivirus Kunjin: construction and applications. J Virol. 71:1497-1505.
27. Khromykh, A. A., M. T. Kenney, and E. G. Westaway. 1998. trans-Complementation of flavivirus RNA polymerase gene NS5 by using Kunjin virus replicon-expressing BHK cells. J Virol. 72:7270-7279.
28. Khromykh, A. A., A. N. Varnavski, and E. G. Westaway. 1998. Encapsidation of the flavivirus Kunjin replicon RNA by using a complementation system providing Kunjin virus structural proteins in trans. J Virol. 72:5967-5977.
29. Khromykh, A. A., P. L. Sedlak, and E. G. Westaway. 2000. cis- and trans-acting elements in flavivirus RNA replication. J Virol. 74:3253-3263.
30. Khromykh, A. A., A. N. Vanavski, P. L. Sedlak, and E. G. Westaway. 2001. Coupling between replication and packaging of flavivirus RNA: evidence derived from the use of DNA-based full-length cDNA clones of Kunjin virus. J Virol. 75:4633-4640.
31. Le, T. T., D. Drane, J. Malliaros, J. C. Cox, L. Rothel, M. Pearse, T. Woodberry, J. Gardner, and A. Suhrbier. 2001. Cytotoxic T cell polyepitope vaccines delivered by ISCOMs. Vaccine. 19:4669-4675.
32. Libraty, D. H., S. Pichyangkul, C. Ajariyakhajom, T. P. Endy, and F. A. Ennis. 2001. Human dendritic cells are activated by dengue virus infection: enhancement by gamma interferon and implications for disease pathogenesis. J Virol. 75:3501-3508.
33. Liljestrom, P., and H. Garoff. 1991. A new generation of animal cell expression vectors based on the Semliki Forest virus replicon. Biotechnology (N Y). 9:1356-1361.
34. Lindenbach, B. D., and C. M. Rice. 1997. trans-Complementation of yellow fever virus NS1 reveals a role in early RNA replication. J Virol. 71:9608-9617.
35. Lindenbach, B. D., and C. M. Rice. 1999. Genetic interaction of flavivirus nonstructural proteins NS1 and NS4A as a determinant of replicase function. J Virol. 73:4611-4621.
36. MacDonald, G. H., and R. E. Johnston. 2000. Role of dendritic cell targeting in Venezuelan equine encephalitis virus pathogenesis. J Virol. 74:914-922.
37. Mandl, C. W., J. H. Aberle, S. W. Aberle, H. Holzinann, S. L. Allison, and F. X. Heinz. 1998. In vitro-synthesized infectious RNA as an attenuated live vaccine in a flavivirus model. Nat Med. 4:1438-1440.
38. Mateo L., J. G., and A. Suhrbier. 2001. Delayed emergence of bovine leukemia virus following vaccination with a protective cytotoxic T cell based vaccine. AIDS Res & Hum Retro. 17 1447-53.
39. Morris-Downes, M. M., B. J. Sheahan, M. N. Fleeton, P. Liljestrom, H. W. Reid, and G. J. Atkins. 2001. A recombinant Semliki Forest virus particle vaccine encoding the prME and NS1 proteins of louping ill virus is effective in a sheep challenge model. Vaccine. 19:3877-3884.
40. Perri, S., D. A. Driver, J. P. Gardner, S. Sherrill, B. A. Belli, T. W. Dubensky, Jr., and J. M. Polo. 2000. Replicon vectors derived from Sindbis virus and Semliki forest virus that establish persistent replication in host cells. J Virol. 74:9802-9807.
41. Pushko, P., M. Parker, G. V. Ludwig, N. L. Davis, R. E. Johnston, and J. F. Smith. 1997. Replicon-helper systems from attenuated Venezuelan equine encephalitis virus: expression of heterologous genes in vitro and immunization against heterologous pathogens in vivo. Virology. 239:389-401.
42. Raju, R., S. V. Subramaniam, and M. Hajjou. 1995. Genesis of Sindbis virus by in vivo recombination of nonreplicative RNA precursors. J Virol. 69:7391-7401.
43. Ramsay, A. J., S. J. Kent, R. A. Strugnell, A. Suhrbier, S. A. Thomson, and I. A. Rainshaw. 1999. Genetic vaccination strategies for enhanced cellular, humoral and mucosal immunity. Immunol Rev. 171:27-44.
44. Rolph, M. S., and I. A. Ramshaw. 1997. Recombinant viruses as vaccines and immunological tools. Curr Opin Immunol. 9:517-524.
45. Scheffe, H. 1959. The analysis of variance. Wiley, New York.
46. Schlesinger, S., and B. G. Weiss. 1994. Recombination between Sindbis virus RNAs. Arch Virol Suppl. 9:213-220.
47. Schlesinger, S., and T. W. Dubensky. 1999. Alphavirus vectors for gene expression and vaccines. Curr Opin Biotechnol. 10:434-439.
48. Seth, A., I. Ournanov, J. E. Schmitz, M. J. Kuroda, M. A. Lifton, C. E. Nickerson, L. Wyatt, M. Carroll, B. Moss, D. Venzon, N. L. Letvin, and V. M. Hirsch. 2000. Immunization with a modified vaccinia virus expressing simian immunodeficiency virus (SIV) Gag-Pol primes for an anamnestic Gag-specific cytotoxic T-lymphocyte response and is associated with reduction of viremia after SIV challenge. J Virol. 74:2502-2509.
49. Sigal, L. J., S. Crotty, R. Andino, and K. L. Rock. 1999. Cytotoxic T-cell immunity to virus-infected non-haematopoietic cells requires presentation of exogenous antigen. Nature. 398:77-80.
50. Thomson, S. A., M. A. Sherritt, J. Medveczky, S. L. Elliott, D. J. Moss, G. J. Fernando, L. E. Brown, and A. Suhrbier. 1998. Delivery of multiple CD8 cytotoxic T cell epitopes by DNA vaccination. J Immunol. 160:1717-1723.
51. Thomson, S. A., S. L. Elliott, M. A. Sherritt, K. W. Sproat, B. E. Coupar, A. A. Scalzo, C. A. Forbes, A. M. Ladhams, X. Y. Mo, R. A. Tripp, P. C. Doherty, D. J. Moss, and A. Suhrbier. 1996. Recombinant polyepitope vaccines for the delivery of multiple CD8 cytotoxic T cell epitopes. J Immunol. 157:822-826.
52. Vamavski, A. N., and A. A. Khromykh. 1999. Noncytopathic flavivirus replicon RNA-based system for expression and delivery of heterologous genes. Virology. 255:366-375.
53. Varnavski, A. N., P. R. Young, and A. A. Khromykh. 2000. Stable high-level expression of heterologous genes in vitro and in vivo by noncytopathic DNA-based Kunjin virus replicon vectors. J Virol. 74:4394-4403.
54. Vassilev, V. B., L. H. Gil, and R. O. Donis. 2001. Microparticle-mediated RNA immunization against bovine viral diarrhea virus. Vaccine. 19:2012-2019.
55. Vignuzzi, M., S. Gerbaud, S. van der Werf, and N. Escriou. 2001. Naked RNA immunization with replicons derived from poliovirus and Semliki Forest virus genomes for the generation of a cytotoxic T cell response against the influenza A virus nucleoprotein. J Gen Virol. 82:1737-1747.
56. Westaway, E. G., J. M. Mackenzie, M. T. Kenney, M. K. Jones, and A. A. Khromykh. 1997. Ultrastructure of Kunjin virus-infected cells: colocalization of NS1 and NS3 with double-stranded RNA, and of NS2B with NS3, in virus-induced membrane structures. J Virol. 71:6650-6661.
57. Wu, S. J., G. Grouard-Vogel, W. Sun, J. R. Mascola, E. Brachtel, R. Putvatana, M. K. Louder, L. Filgueira, M. A. Marovich, H. K. Wong, A. Blauvelt, G. S. Murphy, M. L. Robb, B. L. Innes, D. L. Birx, C. G. Hayes, and S. S. Frankel. 2000. Human skin Langerhans cells are targets of dengue virus infection. Nat Med. 6:816-820.
58. Ying, H., T. Z. Zaks, R. F. Wang, K. R. Irvine, U. S. Kammula, F. M. Marincola, W. W. Leitner, and N. P. Restifo. 1999. Cancer therapy using a self-replicating RNA vaccine. Nat Med. 5:823-827.
59. Perri S., Driver D. A., Gardner J. P., Sherrill S., Belli B. A., Dubensky T. W. Jr and Polo J. M. 2000. Replicon vectors derived from Sindbis virus and Semliki forest virus that establish persistent replication in host cells. J. Virol., 74: 9802-9807.
60. Anraku, I., T. J. Harvey, R. Linedale, J. Gardner, D. Harrich, A. Suhrbier, and A. A. Khromykh. 2002. Kunjin virus replicon vaccine vectors induce protective CD8+T-cell immunity. J Virol. 76:3791-3799.
61. Shi, P. Y., Tilgner, M., and Lo, M. K. 2002. Construction and characterization of subgenomic replicons of New York strain of West Nile Virus. Virology 296:219-233.
62. Yamschikov, V. F., Webgler, G., Perelygin, A. A., Brinton, M. A., and Compans, R. W. 2001. An infectious clone of the West Nile flavivirus. Virology 281:294-304.

1. An expression vector comprising:
(i) a nucleotide sequence encoding a flavivirus replicon that is incapable of producing infectious virus wherein said flavivirus replicon encodes one or more mutated flaviviral non-structural proteins;
(ii) an insertion site for a heterologous nucleic acid;
(iii) a promoter operably linked to the nucleotide sequence encoding the flavivirus replicon; and
(iv) at least one autoprotease-encoding nucleotide sequence.

2. The expression vector of claim 1, comprising two autoprotease-encoding nucleotide sequence, wherein a first of said at least two autoprotease-encoding nucleotide sequences is located 5′ of said insertion site and a second of said at least two autoprotease-encoding nucleotide sequences is located 3′ of said insertion site.

3. The expression vector of claim 1, wherein the autoprotease-encoding nucleotide sequences respectively encode a foot and mouth disease virus 2A autoprotease.

4. The expression vector of claim 1, wherein said mutated flaviviral non- structural protein in (i) is selected from the group consisting of:
(i) a nonstructural protein NS1 having a mutation of Proline 250 to Leucine;
(ii) a nonstructural protein NS2A having a mutation of Alanine 30 to Proline;
(iii) a nonstructural protein NS2A having a mutation of Asparagine 101 to Aspartate; and
(iv) a nonstructural protein NS5 having a mutation of Proline 270 to Serine.

5. The expression vector of claim 1 further comprising nucleotide sequences respectively encoding the first 20 amino acids of C protein (C20) and the last 22 amino acids of E protein (E22).

6. The expression vector of claim 1 wherein the replicon comprises one or more nucleotide sequences respectively encoding a wild-type nonstructural flaviviral protein selected from NS1, NS2A, NS2B, NS3, NS4A, NS4B and NS5.

7. The expression vector of claim 1, wherein the replicon is derived from Kunjin virus.

8. The expression vector of claim 1, which is SP6KUNrep5 or pKUNrep5.

9. The expression vector of claim 2, which is SP6KUNrep6 or pKUNrep6.

10. An expression construct comprising:
(i) a nucleotide sequence encoding a flavivirus replicon that is incapable of producing infectious virus wherein said flavivirus replicon encodes one or more mutated flaviviral non-structural proteins;
(ii) a heterologous nucleic acid;
(iii) a promoter operably linked to the nucleotide sequence encoding the flavivirus replicon; and
(iv) at least one autoprotease-encoding nucleotide sequence.

11. The expression construct of claim 10 comprising at least two autoprotease-encoding nucleotide sequences, wherein a first of said at least one autoprotease-encoding nucleotide sequences is located 5′ of said heterologous nucleic acid and a second of said at least one autoprotease-encoding nucleotide sequences is located 3′ of said heterologous nucleic acid.

12. The expression vector of claim 10, wherein the autoprotease-encoding nucleotide sequences respectively encode a foot and mouth disease virus 2A autoprotease.

13. The expression construct of claim 10, wherein said mutated flaviviral non-structural protein in (i) is selected from the group consisting of:
(i) a nonstructural protein NS1 having a mutation of Proline 250 to Leucine;
(ii) a nonstructural protein NS2A having a mutation of Alanine 30 to Proline;
(iii) a nonstructural protein NS2A having a mutation of Asparagine 101 to Aspartate; and
(iv) a nonstructural protein NS5 having a mutation of Proline 270 to Serine.

14. The expression construct of claim 10 further comprising nucleotide sequences respectively encoding the first 20 amino acids of C protein (C20) and the last 22 amino acids of E protein (E22).

15. The expression construct of claim 10 further comprising nucleotide sequences respectively encoding a flaviviral wild-type nonstructural protein selected from NS1, NS2A, NS2B, NS3, NS4A, NS4B and NS5.

16. The expression construct of claim 15, wherein the flaviviral replicon is derived from Kunjin virus.

17. The expression construct of claim 10, wherein the heterologous nucleic acid encodes an immunogenic protein.

18. The expression construct of claim 17, wherein the immunogen is a murine polyepitope protein having one or more of the epitope sequences YPHFMPTNL, RPQASGVYM, TYQRTRALOV, SYIPSAEKI and SIINFEKL.

19. The expression construct of claim 17, wherein the immunogenic protein is HIVgag protein.

20. The expression construct of claim 10 which is an RNA construct

21. The expression construct of claim 20 which is RNALeuMpt, RNAProMpt or KUNRNAgag.

22. The expression construct of claim 10 which is a DNA construct.

23. The expression construct of claim 22 which is DNALeuMpt, DNAProMpt, KUNDNAgag.

24. A host cell which comprises the expression construct of claim 10.

25. An expression system comprising:
(i) the expression construct of claim 10; and
(ii) at least another expression construct that is capable of expressing one or more proteins that facilitate packaging of said expression construct into flavivirus virus like particles (VLPs).

26. The expression system of claim 25, wherein the expression construct of claim 10 is an RNA construct.

27. The expression system of claim 25, wherein said another expression construct is derived from semliki forest virus (SFV) or sindbis virus (SIN).

28. The expression system of claim 27, which is derived from SFV.

29. The expression system of claim 27, further comprising a mutated nsP2 gene of SFV.

30. The expression system of claim 29, wherein the mutated nsP2 gene of SFV encodes a substitution of proline for leucine 713.

31. The expression system of claim 30, wherein said at least another expression construct is SFVMEC/L713P/Neo, pSFV3L713PLacZNeo or SFVMEC/L713P.

32. A host cell comprising said at least another expression construct selected from SFVMEC/L713P/Neo, pSFV3L713PLacZNeo or SFVMEC/L713P.

33. The host cell of claim 32 which is a BHK21 cell stably transfected with SFVMEC/L713P/Neo or pSFV3L713PLacZNeo.

34. The host cell of claim 32, which further comprises the expression construct of claim 10.

35. The host cell of claim 34, which produces one or more VLPs that comprise an RNA that encodes an immunogenic protein.

36. A pharmaceutical composition comprising a flaviviral DNA expression construct from which RNA is transcribable in an animal cell and a pharmaceutically-acceptable carrier, diluent or excipient, wherein the transcribable RNA encodes:
(i) a flavivirus replicon that is incapable of producing infectious virus; and
(ii) an immunogenic protein.

37. A pharmaceutical composition comprising an RNA that is transcribable from a flaviviral DNA expression construct and a pharmaceutically-acceptable carrier, diluent or excipient, which RNA encodes:
(i) a flavivirus replicon that is incapable of producing infectious virus; and
(ii) an immunogenic protein.

38. The pharmaceutical composition of claim 36 or 37, wherein the flaviviral DNA expression construct comprises:
(i) a DNA sequence encoding a flavivirus replicon that is incapable of producing infectious virus wherein said flavivirus replicon encodes one or more mutated flaviviral non-structural proteins;
(ii) a heterologous DNA encoding said immunogenic protein;
(iii) a promoter operable in an animal cell and which is operably linked to the DNA sequence encoding the flavivirus replicon; and
(iv) at least one autoprotease-encoding nucleotide sequence.

39. The pharmaceutical composition of claim 36 or 37 wherein the flavivirus is Kunjin virus.

40. The pharmaceutical composition of claim 36 or 37, which is a vaccine.

41 The vaccine of claim 40, which is capable of eliciting a protective T cell response upon administration to an animal.

42. The vaccine of claim 41, wherein the T cell response is a CTL response.

43. The vaccine of claim 42, wherein the CTL response is characterized by a long-term effector CD8+T cell response.

44. The vaccine of claim 43 wherein the immunogen is a viral protein.

45. The vaccine of claim 44 wherein the viral protein is HIVgag.

46. The vaccine of claim 40, wherein the animal is a mammal.

47. The vaccine of claim 46, wherein the mammal is a human.

48. A vaccine comprising one or more VLPs produced by the expression system of claim 20, wherein said VLPs include an RNA that encodes said immunogenic protein.

49. The vaccine of claim 48, wherein the immunogenic protein is a viral protein.

50. The vaccine of claim 49, wherein the viral protein is HIVgag.

51. A method of immunizing an animal including the step of administering the vaccine of claim 40 or claim 48 to said animal to thereby induce immunity in said animal.

52. The method of claim 51, wherein immunity is antibody-mediated.

53. The method of claim 51, wherein immunity is cell mediated immunity.

54. The method of claim 53, wherein immunity is T cell mediated immunity.

55. The method of claim 54, wherein T cell immunity is characterized by a CD8+cytotoxic T lymphocyte (CTL) response.

56. The method of claim 55, wherein T cell immunity is characterized by induction of a long-term effector CD8+CTL response.

57. The method of claim 56, wherein, T cell immunity is characterized by a CD4+T cell response.

58. The method of claim 51, wherein immunity to viral infection is induced in said animal.

59. The method of claim 51 wherein tumour immunity is induced in said animal.

60. The method of claim 59, wherein immunity to melanoma is induced in said animal.

61. The method of claim 51, wherein the animal is a mammal.

62. The method of claim 60, wherein the mammal is a human.

63. A packaging construct selected from the group consisting of SFVMEC/L713P/Neo, pSFV3L713PLacZNeo or SFVMEC/L713P.

64. A host cell comprising the packaging construct of claim 63.

65. The host cell of claim 64 which is a BHK21 cell stably transfected with SFVMEC/L713P/Neo or pSFV3L713PLacZNeo.
